SEQUENCE LISTING (14 sequences)

SEQ ID NO: 1 - protein, 6 aa, Artificial
  Enzyme substrate
PVGLIG

SEQ ID NO: 2 - protein, 9 aa, Artificial
  Enzyme substrate
RPLALWRSC

SEQ ID NO: 3 - DNA, 20 bp, Artificial
  Synthetic construct
tccatgacgttcctgacgtt

SEQ ID NO: 4 - protein, 22 aa, Artificial
  HIV antigen
CGCRIQRGPGRAFVTIGKCGCG

SEQ ID NO: 5 - protein, 7 aa, Artificial
  Enzyme substrate
RRRRGCG

SEQ ID NO: 6 - protein, 11 aa, Artificial
  Enzyme substrate
RPLALWRSGCG

SEQ ID NO: 7 - protein, 10 aa, Artificial
  enzyme substrate
GFQGVQFAGF

SEQ ID NO: 8 - protein, 10 aa, Artificial
  Enzyme substrate
GFGSVGFAGF

SEQ ID NO: 9 - protein, 10 aa, Artificial
  Enzyme substrate
GLVGGAGAGF

SEQ ID NO: 10 - protein, 10 aa, Artificial
  Enzyme substrate
GGFLGLGAGF

SEQ ID NO: 11 - protein, 10 aa, Artificial
  Enzyme substrate
GFGSTFFAGF

SEQ ID NO: 12 - protein, 6 aa, Artificial
  Enzyme substrate
PLGLAR

SEQ ID NO: 13 - protein, 7 aa, Artificial
  Enzyme substrate
PMALWMR

SEQ ID NO: 14 - protein, 6 aa, Artificial
  Enzyme substrate
PMGLRA

(19) United States
(12) Patent Application Publication
(10) Pub. No.: US 2008/0014149 A1
(43) Pub. Date: Jan. 17, 2008

(54) Methods and Compositions for Imaging and Biomedical Applications

(76) Inventors: Niren Murthy, Atlanta, GA (US); Jihua Hao, Marietta, GA (US); Amy R. Guinn, Atlanta, GA (US); Stephen C. Yang, Smyrna, GA (US); Michael J. Hefferman, Atlanta, GA (US); Bali Pulendran, Atlanta, GA (US)

Correspondence Address: TROUTMAN SANDERS LLP, 600 PEACHTREE STREET , NE, ATLANTA, GA, 30308, US

(21) Appl. No.: 11/578,261

(22) PCT Filed: Apr. 13, 2005
(86) PCT No.: PCT/US05/12571

§ 371 Date: Oct. 12, 2006

Related U.S. Application Data
(60) Provisional application No. 60/561,317, filed on Apr. 12, 2004.
(60) Provisional application No. 60/617,550, filed on Oct. 8, 2004.
(60) Provisional application No. 60/658,050, filed on Mar. 2, 2005.

(51) Int. Cl. A61B 5/055

(52) U.S. Cl. 424/9.36

(57) ABSTRACT

The present invention is directed to detectable compositions and methods for making and using such compositions. Detectable compositions comprise detectable constructs comprising a detectable agent. Due to the actions of a specific bioactivity in vivo or in vitro, the detectable construct is altered in some manner so that the detectable agent is detected. The present invention provides diagnostic imaging agents such as for MRI and optical imaging, which are used for sensitive detection of a specific bioactivity within a tissue. The present invention comprises methods and compositions for biocleavable or biodegradable compositions for carrying and releasing active agents for therapeutic or other medical uses. The methods and compositions of the present invention further comprise micelle compositions. The active agents of the present invention may comprise drugs, vaccines, and imaging agents.

### TECHNICAL FIELD

The present invention discloses methods and compositions for targeted delivery of active agents and detection of bioactivity for therapeutic or other medical uses.

### BACKGROUND OF THE INVENTION

The ability to image and detect biological activity in a whole organism is a goal of many diagnostic systems. Several imaging systems are routinely used to diagnose physical conditions. Scanning of whole organisms, organs or sections can be accomplished by techniques used in magnetic resonance imaging (MRI), ultrasound scanning, x-ray, CAT (computer axial tomography), nuclear radioisotope tracking, PET (positron emission tomography) scanning, ultraviolet, visible and infrared light imaging. In an effort to image specific tissues or detect specific activities, targeted scanning or contrast materials have been developed.

Among the choices for imaging living organisms is CAT or CT (computed tomography scanning. A computed tomography (CT) scan uses X-rays to produce detailed pictures of structures inside the body. A CT scanner directs a series of X-ray pulses through the body. Each X-ray pulse lasts only a fraction of a second and represents a “slice” of the organ or area being studied. The slices or pictures are recorded on a computer and can be saved for further study or printed out as photographs.

A scan that is capable of measuring some types of bioactivity is the PET scan. Positron emission tomography (PET scan) is a test that combines CT and nuclear scanning. During a PET scan, a radioactive substance called a tracer is combined with a chemical (such as glucose); this mixture is generally injected into a vein (usually in the arm) but on occasion may be inhaled. The tracer emits tiny positively charged particles (positrons) that produce signals. The chemical substance and radioactive tracer chosen for the test vary according to which area of the body is being studied. A camera records the tracer's signals as it travels through the body and collects in organs. A computer then converts the signals into three-dimensional images of the examined organ. The three-dimensional views can be produced from any angle and provide a clear view of an abnormality. Compared to CT scans and MRI scans, PET produce less-detailed pictures of an organ. A PET scan is often used to detect and evaluate cancer, such as of the lung or breast. It also can be used to evaluate the heart's metabolism and blood flow and examine brain function.

Magnetic resonance imaging (MRI) is a diagnostic and research procedure that uses high magnetic fields and radio-frequency signals to produce images. The most abundant molecular species in biological tissues is water. It is the quantum mechanical “spin” of the water proton nuclei that gives rise to the signal. In MRI the sample to be imaged is placed in a static magnetic field (1-12 Tesla) and the spins are excited with a pulse of radio frequency (RF) radiation to produce a net magnetization. Various magnetic field gradients and other RF pulses then act on the spins to code spatial information into the recorded signals. MRI is used to generate structural information in three dimensions in relatively short time spans.

MR images are generally displayed on a gray scale with black the lowest and white the highest measured intensity. Though variations in water concentration may add to the contrast in MR images, the key to definitive images is the rate of change of the magnetization at the time of measurement on local environment that is the source of image intensity variation in MRI. Two characteristic relaxation times, T₁ and T₂, govern the rate at which the magnetization can be accurately measured. T₁ is the exponential time constant for the spins to decay back to equilibrium after being perturbed by the radiofrequency (RF) pulse. In order to increase the signal-to-noise ratio a typical MR imaging scan (RF & gradient pulse sequence and data acquisition) is repeated at a constant rate for a predetermined number of times and the data averaged. The signal amplitude recorded for any given scan is proportional to the number of spins that have decayed back to equilibrium since the previous scan. Thus, regions with rapidly decaying spins (i.e. short T₁ values) will recover all of their signal amplitude between successive scans.

The measured intensities in the final image will accurately reflect the spin density (i.e. water content). Regions with long T₁ values compared to the time between scans will progressively lose signal until a steady state condition is reached and will appear as darker regions in the final image. Changes in T₂ (spin-spin relaxation time) result in changes in the signal linewidth (shorter T₂ values) yielding larger linewidths. In extreme situations the linewidth can be so large that the signal is indistinguishable from background noise. The water relaxation characteristics vary from tissue to tissue, thus producing measurable differences in T₁ or T₂ measurements, which provides the contrast which allows the discrimination of tissue types.

The physical parameters measured for other types of scanning are also well known and though some can be used for detecting biological activity, none of the current imaging systems can discriminate specific activities in methods that are currently widely available. For example, cost and patient safety are an issue when detection systems require injection of radionuclides. What is needed are detectable agents, also known as contrast agents that are sensitive and can be targeted to specific sites or activities in the body. In particular, there is a need for detectable agents that provide signal enhancement or are detectable in response to specific cellular or tissue activities. Such detectable agents would be useful in diagnostic and prognostic methods. Additionally, targeted biological constructs are needed that can provide detectable agents, pharmaceuticals, nutriceuticals and immunological agents such as vaccines to specific sites.

The ability to specifically deliver and release active agents for therapeutic or other medical uses is needed for pharmaceutical agents, vaccines, imaging agents, biologicaly active peptides, and recombinant protein therapeutics. Desired characteristics for delivery vehicles include small size, biodegradability, good loading capacity, biocompatibility, and the ability to be specifically directed to a target site. Despite significant developments in the area of delivery vehicles, there remain specific challenges that limit the effectiveness, specificity and broad applicability of many of the currently available technologies.

Further, there is a need for the development of vaccines against AIDS, hepatitis C, emerging viruses, and cancer. Traditional vaccination strategies for infectious disease has typically involved use of live attenuated viruses. Unfortunately, this approach has been largely ineffective against many viral diseases and is not applicable to diseases such as cancer. Vaccines based upon protein or peptide antigens have considerable promise due to their low toxicity and widespread applicability to not only infectious disease, but also as a new approach to diseases such as cancer. However, delivery problems have limited the clinical success of protein- and peptide-based vaccines.

Direct administration of drugs, or other active agents, particularly hydrophobic drugs, either orally, parenterally, or intravenously, often results in poor delivery of the drug to the desired target site. However, increasing the hydrophobic character of a drug also increases the membrane permeability at the site of action of the drug, and oftentimes, the effectiveness of a drug. One result of specific delivery of a drug is the ability to use smaller quantities of drug and reducing the risks of harmful side effects arising from general distribution of the drug throughout the body. For example, formulation of hydrophobic drugs for oral administration represents a significant challenge to maintaining the drug in solution long enough to be able to cross the intestinal wall. Frequently, such drugs aggregate in the intestine and passed out the body with fecal material. Intravenous administration of largely hydrophobic drugs can also result in aggregates once dispersed in the circulatory system, and can cause embolization of blood capillaries before the drug reaches the target site. One approach that has been attempted is the use of liposomes for poorly soluble drugs. Although liposomes can entrap poorly soluble drugs in the hydrophobic bilayer, they have poor loading capacity because of possible membrane destabilization. Further, liposomes do not enhance in any way the specific delivery and release of drug at the target site.

Micelles are composed of many amphiphilic molecules which self-assemble under the appropriate conditions. In water or aqueous solutions, hydrophobic portions of the amphiphilic molecules form the core of the micelle. The core of a micelle many be used as a cargo space for poorly soluble therapeutic agents. Hydrophilic portions of the amphiphilic molecules form the micellar corona. However, under physiological conditions, many micelles do not remain intact and essentially dissociate into individual amphiphilic molecules. Under such conditions, any cargo retained within the core of the micelle is exposed to the aqueous environment. If the cargo were a hydrophobic molecule, it would likely precipitate or aggregate and be rendered ineffective. Further, micelles as such do not necessarily possess properties that target them, and their cargo, to specific sites of action.

What is needed are methods and compositions for delivery vehicles that can be targeted to specific sites to effectively deliver or release active agents, including contrast agents, drugs, peptides, nucleic acids, immunogens or proteins. In particular, a need exists for methods and compositions for micelles that are generally stable in the body, but are capable of delivery or release of active agents at the desired site of action. Such micellar delivery vehicles would increase the ability of the active agents to be transported into or within the body, and once in the body, to be transported more specifically to the site of action. Further, a need exists for micellar delivery vehicles for active agents that are poorly soluble in aqueous solution. What is also needed are delivery vehicles that are effective as vaccines, such as protein- and peptide-based vaccines or other immunogenic materials such as immunostimulatory DNA fragments.

### SUMMARY

The present invention is directed to detectable compositions and methods for making and using such compositions. Detectable compositions comprise detectable constructs comprising a detectable agent. Due to the actions of a specific bioactivity in vivo or in vitro, the detectable construct is altered in some manner so that the detectable agent is detected. The present invention provides diagnostic imaging agents such as for MRI and optical imaging, which are used for sensitive detection of a specific bioactivity within a tissue. In the presence of the bioactivity, or because of actions on the detectable composition, there is a change that increases the signal (or image) contrast between tissues which contain the targeted bioactivity and those which do not, which thus reflects the presence of the targeted bioactivity.

An aspect of the present invention comprises a detectable construct comprising a detectable agent, such as a paramagnetic metal ion, radioactive ion, or other detectable compound, bound to the construct. The detectable agent may also comprise a chelator of the paramagnetic or radioactive metal ion, wherein the chelator molecule is covalently bonded to the detectable construct, and the chelator may or may not be modified. In one aspect, the invention provides detectable agents, such as MRI contrast agents, comprising a paramagnetic metal ion including, but not limited to, Gd(III), Fe(III), Mn(II), Yt(III), Cr(III) and Dy(III). In another aspect, the invention provides detectable constructs which may be used as MRI contrast agents comprising a first paramagnetic metal ion bound to a first chelator, and at least a second paramagnetic metal ion bound to a second chelator. A detectable construct may comprise at least two detectable agents comprising paramagnetic metal ions, each bonded with a chelator molecule, and the detectable agents may flank one or more regions such as a reaction region or linking region. A detectable composition may comprise subcompositions comprising detectable constructs wherein each subcomposition comprises a detectable construct having detectable agent, such as a paramagnetic metal ion with a chelator molecule, and the detectable construct may have one or more regions such as a reaction region or a linking region.

The present invention also comprises methods of imaging a cell, tissue, animal or human comprising administering a detectable composition comprising a detectable agent of the invention to a cell, tissue, animal or patient for diagnostic or prognostic detection of a bioactivity and rendering an image of the cell, tissue, animal or human patient. The present invention also comprises detectable constructs comprising micelles.

The present invention comprises micellar compositions and methods of making and using such micellar compositions for delivery of active agents, such as pharmaceuticals, imaging agents, immunogens or nucleic acids, to specific or target sites in a body. Micellar compositions comprise micelles comprising amphiphilic polymeric molecules comprising pendant side chains capable of bonding with cross-linking molecules. Cross-linking molecules function to stabilize the micelle and may also function to target the micelle to a particular site, provide a reaction region or be an active agent.

### BRIEF DESCRIPTION OF DRAWINGS

FIG. 1 is a schematic showing the preparation of peptide crosslinked micelles useful for vaccines.

FIG. 2 is a reaction scheme for the preparation of PEG-PLL(thiopyridyl).

FIG. 3 is a NMR spectra of PEG-PLL(thiopyridyl).

FIG. 4 is a graph of dynamic light scattering data obtained for micelles prepared using PEG-PLL(thiopyridyl).

FIG. 5 is a graph of dynamic light scattering data obtained for peptide crosslinked micelles prepared using PEG-PLL(thiopyridyl).

FIG. 6 is a comparison of UV spectra obtained from peptide crosslinked micelles treated under different conditions.

FIG. 7 is a graph show the rate of reaction of peptide with PEG-PLL(thiopyridyl).

FIG. 8 is a graph comparing the rate of peptide release from peptide crosslinked micelles treated with glutathione.

FIG. 9 shows results obtained using agarose gel electrophoresis for analysis of peptide crosslinked micelles treated under different conditions.

FIG. 10 shows results obtained using agarose gel electrophoresis for analysis of peptide crosslinked micelles treated under different conditions.

FIG. 11 is a schematic showing the preparation of peptide crosslinked micelles comprising a detectable construct.

FIG. 12 is a schematic comparing two different detectable constructs.

FIG. 13 is a graph showing the T₂ obtained from a detectable micellar construct treated with MMP-7

FIG. 14 is a schematic showing the preparation and use of a detectable construct.

FIG. 15 is a schematic showing the preparation and use of a detectable construct.

FIG. 16 is a schematic showing the synthetic scheme for preparation of a detectable construct.

FIG. 17 is a schematic showing the synthetic scheme for preparation of a detectable construct.

FIG. 18 is a schematic showing the synthetic scheme for preparation of a detectable construct.

FIG. 19 is a graph showing data obtained using a detectable construct.

FIG. 20 is a schematic showing the synthetic scheme for preparation of a detectable construct.

FIG. 21 is a schematic showing the synthetic scheme for preparation of a detectable construct.

FIG. 22 is a schematic showing the synthetic scheme for preparation of a detectable construct.

FIG. 23 is a schematic showing the synthetic scheme for preparation of a detectable construct.

FIG. 24 is a structure of a detectable construct.

FIG. 25 is a graph showing results obtained with a detectable construct treated with trypsin.

FIG. 26 is a schematic showing the synthetic scheme for preparation of a detectable construct.

FIG. 27 is a schematic showing the synthetic scheme for preparation of a detectable construct.

FIG. 28 is a schematic showing the synthetic scheme for preparation of a detectable construct.

### DETAILED DESCRIPTION

The present invention comprises detectable compositions and methods of making and using such detectable compositions. The detectable compositions comprise at least one detectable agent that can be detected by imaging techniques including but not limited to, MRI (Magnetic Resonance Imaging), optical imaging, SPECT, PET and Radionuclide imaging. The methods of using such detectable compositions include detecting bioactivity in a body, tissue, cells, including intracellularly or extracellularly, and can be used in applications that are in vivo, ex vivo and in vitro. The detection of bioactivity provides methods of diagnosis and prognosis of physiological states including, but not limited to, health or disease, function or dysfunction, and growth or unwanted growth of tissues and cells.

An aspect of the present invention comprises compositions comprising a detectable agent. A detectable agent is an element or molecule that can be detected by imaging methods. Many such detectable agents are known to those skilled in the art for use in imaging systems such as MRI, optical scanning, PET scans, CT scans, infrared or visible scanning methods. The present invention contemplates use of known detectable agents and others that can be used in the detectable constructs disclosed herein. An example of a detectable agent is a paramagnetic ion and an associated chelate molecule, including but not limited to DTPA and DOTA. The chelator molecule may be chemically modified for particular purposes, such as to alter solubility, diffusion rates or other modifications. Modification of such chelate molecules are known in the art.

An aspect of the present invention comprises a detectable composition comprising a detectable construct comprising at least two detectable agents, wherein the detectable agents are part of the same detectable construct, and the detectable agents are the same detectable agent. The present invention comprises detectable compositions comprising a detectable construct comprising at least two detectable agents, wherein the detectable agents are a part of the same detectable construct and the detectable agents are not the same. Detectable agents are not the same, or alternatively, are referred to herein as different, when the agents are not identical chemical elements, have different linking or chelating molecules, or are detected by different physical or chemical means such as one agent is detected using MRI and the other is detected using optical means. Further, detectable agents are not the same and are different, when the measured parameter or parameters of one agent is not the same as the physical parameter or parameters of another agent. For example, two detectable agents are not the same and are different when they are chemically different such as when one detectable agent comprises the element Dy and another detectable agent comprises the element Gd or wherein the measured parameter of one agent is the T₁ relaxation time and the measured parameter of the other agent is the T₂ relaxation time.

An aspect of the present invention comprises a detectable composition comprising a detectable construct wherein the detectable construct comprises a reaction region. The reaction region is a portion of the construct that is acted on chemically or physically by a biological molecule or chemical process of a living organism. The physical or chemical action by a biological molecule or chemical process of a living organism is herein referred to as bioactivity. The reaction region is altered by the bioactivity. Detection by the present invention comprises detecting the bioactivity or lack of bioactivity by measuring the effects of the alteration the reaction region. For example, the reaction region may be the substrate for an enzyme such that when the detectable construct encounters the enzyme, the substrate or reaction region is acted on by the enzyme. The reaction region may be a chemically reactive combination of elements, such as a disulfide bond, that when in the appropriate environment, the disulfide bond is cleaved resulting in an alteration of the reaction region and a change in the detectable construct such that the measured parameters are changed or detected. The reaction region may be one of a pair of binding partners, such that the binding of the binding partner incorporated in the detectable construct with the other binding partner found in the body alters the reaction region, and results in a change in the detectable construct that is measurable

An aspect of the compositions of the present invention comprises detectable constructs comprising a linking region. A linking region is a region that may be bonded to the detectable agent or may be provided between a detectable agent and another section of the detectable construct. For example, a detectable construct may comprise a detectable agent bonded to a linking region, or a construct may comprise a detectable agent bonded to a linking region which is bonded at another site to a reaction region. A detectable construct may comprise a detectable agent bonded to a reaction region and the reaction region is bonded at a separate site to a linking region and the linking region is bonded at a second site to another detectable agent. The linking region may be one or more atoms in length and may comprise multiple compounds such as polymeric compounds. The linking region may function to provide size or shape to the detectable construct to aid in the function or delivery of the detectable construct. For example, the linking region may provide a length to the detectable construct such that the construct is not taken up by cells and is maintained in the extracellular space of the tissue or organism. Alternatively, the linking region may provide a size or shape to the detectable construct such that the construct is taken up by cells and is acted on by intracellular processes. Examples of functionality that the linking region may provide are that the detectable construct has an adequate size, shape or charge such that the construct would remain in solution, would be maintained in the blood stream or lymphatic system, would cross the blood-brain barrier, would not be taken up by cells such as macrophages or liver cells, or other controls for location maintenance of the detectable construct. Such size, shape and charge characteristics are known to those skilled in the art.

An aspect of the present invention comprises detectable compositions comprising a combination of two or more subcompositions wherein each of the subcompositions comprises a detectable agent. A subcomposition may comprise a detectable construct comprising one or more detectable agents that are the same or are different from another agent in the same construct, or the detectable agents are the same or are different from the detectable agents present in the other subcomposition. For example, a detectable composition may comprise a subcomposition having a detectable construct comprising detectable agent A, and a second subcomposition comprises a detectable construct comprising detectable agent B, wherein A and B are different detectable agents. Another example may be a composition comprising a subcomposition having a detectable construct comprising detectable agents A and B, and another subcomposition comprises a detectable construct comprising detectable agents A and C. A further example may be a composition comprising a subcomposition having a detectable construct comprising detectable agents A and B, and another subcomposition comprises a detectable construct comprising detectable agents C and D. Alternatively, each detectable agent A, B, C, and D may each be provided on a separate detectable construct in an individual subcomposition. Additionally, one or more of the detectable constructs may have a reaction region. Further, one or more of the detectable constructs may have a linking region. The present invention contemplates these and other combinations of detectable constructs in detectable compositions.

The compositions of the present invention can be used for diagnosis and prognosis of health or disease in a human or animal and methods of imaging living organisms or in vitro imaging of living cultures can be used. One method of providing a detectable image using the present invention in MRI techniques comprises using the ratio of T₂/T₁ to generate the image. In general, the method comprises administering an effective amount of a detectable composition comprising at least one detectable construct. The detectable construct may comprise two detectable agents. Alternatively, the detectable composition may comprise two or more detectable constructs. For example, after administration of a detectable composition, for one detectable agent, the T₁ is measured, and for another detectable agent, the T₂ is measured and one or more measurements are made over a specified time range. The ratio of T₂/T₁ is determined. At least one detectable construct has a region, a reaction region, that is acted on or effected by a biological activity, or bioactivity, such that the starting relationship between the T₂ measured detectable agent and the T₁ measured detectable agent is altered which results in a change in the T₂/T₁ ratio and thus, a change in the signal at the site of the bioactivity. For example, if the T₁ and T₂ detectable agents are found on one detectable construct, the reaction region may be acted on by an enzyme, the bioactivity to be measured. The enzyme may cleave the detectable construct and physically separate the T₁ and T₂ detectable agents which alters each agent's measured signal, and thus, alters the T₂/T₁ ratio and the image obtained. In areas where the enzyme activity is not present, the T₂/T₁ ratio would not be altered in this manner. Alternatively, the two detectable agents may be present on two separate detectable constructs, where at least one of the detectable constructs has a reaction region. Enzyme activity would effect the reaction region, again, changing the T₂/T₁ ratio and the image. The ratiometric methodology allows for the calculation of the percentage of injected detectable agent that is cleaved. This enables the calculation of enzyme activities and ultimately enzyme concentration. A key benefit of the ratiometric strategy over other enzyme probes is that is measurements are independent of tissue concentration heterogeneities. For example, other enzyme cleavable systems usually result in changing T1 and T2, and it is impossible to distinguish between a region of tissue that simply has more contrast from a region of tissue that has the contrast agent cleaved.

Embodiments of the detectable compositions and methods of the present invention are herein described and are in no way to be considered as limiting the present invention to the particularly described constructs, compositions or applications. In general, the detectable constructs of the present invention have one or more specific sites, the reaction regions, in their structure that can become modified in vivo by a specific bioactivity and the modified form can be detected. Generally, the image contrast between normal and abnormal tissue is seen when the bioactivity in one of the tissues is higher than that in the other. If abnormal tissue expresses a greater concentration of bioactivity than normal tissue, then abnormal tissue will modify more of the detectable construct than will normal tissue. Conversely, if the abnormal tissue expresses the lesser bioactivity, then abnormal tissue will have a relatively lower concentration of bioactivated detectable construct.

The term “bioactivity” includes changes in pH, redox potential, concentration of reactive species such as free radicals, or the presence or level of enzymes or biomolecules (including RNA enzymes) that can modify or alter the reaction region of a detectable construct. A “bioactivity” can comprise two or more types of biomolecules that together or sequentially cause modification or alteration of the reaction region. More than one modification or alteration can occur to the reaction region.

The regions of the detectable constructs of the present invention can be arranged in a variety of positions with respect to each other, and such regions include the detectable agent, the reaction region, and the linking region. While these regions can exist without any specific boundaries between them, it is convenient to conceptualize them as separate units of the molecule. Detectable constructs may comprise one or more of each type of region, for example, a detectable construct may comprise one or more reaction regions, one or more detectable agents, and one or more linking regions. Additionally, detectable constructs may not contain a region, for example, a detectable construct comprises a detectable agent and a reaction region.

The compositions and methods of the present invention can be used to detect, diagnose, or follow the progression (prognosis) of cancerous or precancerous changes, or other changes, in the cells or tissues of living organisms or follow the effectiveness of treatment regimens by measuring cellular changes or other bioactivity. As more specific cellular markers are found, the compositions of the present invention can be used to detect those cellular markers or the activity of such cellular markers, whether on the outer membrane surfaces, on internal membranes, internally or externally of the cells. For example, the early diagnosis of cancer is a major medical challenge. Current diagnosis methods generally identify tumors only after they have reached a size that is difficult to manage and thus new diagnostic strategies are greatly needed. Numerous enzymes, such as matrix metalloproteinase 9 (MMP-9), are over-expressed during the early stages of tumor development and are generally absent in normal tissue (Ramos-DeSimone 1999, Tutton 2003, Giannelli 2002, Chambers 2002, and Coussens 2002). The imaging of these enzymes in vivo leads to the early detection of cellular changes and improve the treatment of cellular changes, such as cancer. The present invention comprises methods and compositions for imaging enzymes that are indicators of cellular changes such as cancer.

Compositions of the present invention that are described and contemplated herein can be used to image enzymes, through the enzyme bioactivity on the reaction region. Enzymes that are contemplated by the present invention comprise those listed herein and enzymes having a substrate, cofactor, coenzyme or other molecule that can be acted on by the enzyme wherein the molecule to be acted on can be incorporated in a detectable construct of the present invention. Suitable classes of enzymes which can be detected by the present invention include, but are not limited to, hydrolases such as proteases, carbohydrases, lipases and nucleases; isomerases such as racemases, epimerases, tautomerases, or mutases; transferases, kinases and phophatases. As will be appreciated by those skilled in the art, the potential list of suitable enzyme targets is quite large. Enzymes associated with the generation or maintenance of arterioschlerotic plaques and lesions within the circulatory system, inflammation, wounds, immune response, tumors, may all be detected using the present invention. Enzymes such as lactase, maltase, sucrase or invertase, cellulase, alpha-amylase, aldolases, glycogen phosphorylase, kinases such as hexokinase, proteases such as serine, cysteine, aspartyl and metalloproteases may also be detected, including, but not limited to, trypsin, chymotrypsin, and other therapeutically relevant serine proteases such as tPA and the other proteases of the thrombolytic cascade; cysteine proteases including: the cathepsins, including cathepsin B, L, S, H, J, N and O; and calpain; metalloproteinases including MMP-1 through MMP-10, particularly MMP-1, MMP-2, MMP-7 and MMP-9; and caspases, such as caspase-3, -5, -8 and other caspases of the apoptotic pathway, and interleukin-converting enzyme (ICE). Similarly, bacterial and viral infections may be detected via characteristic bacterial and viral enzymes. The lists of enzymes herein are not meant to be limiting. Once the target enzyme is identified or chosen, enzyme substrate moieties can be designed using well known parameters of enzyme substrate specificities, or similar design techniques for other molecules required or acted on by the target enzyme. For example, when the enzyme target substance is a protease, the reaction region of the detectable construct may be a peptide or polypeptide which is capable of being cleaved by the target protease. By “peptide” or “polypeptide” herein is meant a compound of about 2 to about 30 amino acid residues covalently linked by peptide bonds. Reaction regions may comprise utilize polypeptides from about 2 to about 30 amino acids. The amino acids may be naturally occurring amino acids, or amino acid analogs and peptidomimitic structures. Under certain circumstances, the peptide may be only a single amino acid residue. Examples of such enzyme and detectable construct reaction regions include, but are not limited to, cat B and GGGF; cat B and GFQGVQFAGF (SEQ ID NO: 7); cat B and GFGSVGFAGF (SEQ ID NO: 8); cat B and GLVGGAGAGF (SEQ ID NO: 9); cat B and GGFLGLGAGF (SEQ ID NO: 10); cat D and GFGSTFFAGF (SEQ ID NO: 11); caspase-3 and DEVD; MMP-7 and PELR; MMP-7 and PLGLAR (SEQ ID NO: 12); MMP-7 and PGLWA-(D-arg); MMP-7 and PMALWMR (SEQ ID NO: 13); and MMP-7 and PMGLRA (SEQ ID NO: 14).

Other enzyme and detectable construct reaction regions are contemplated by the present invention. When the enzyme target is a carbohydrase, the detectable construct reaction region will be a carbohydrate group which is capable of being cleaved by the target carbohydrase. For example, when the enzyme target is lactase or beta-galactosidase, the enzyme substrate detectable construct reaction region is lactose or galactose. Similar enzyme/detectable construct reaction region pairs include sucrase/sucrose, maltase/maltose, and alpha-amylase/amylose. In addition, the detectable constructs may comprise the addition of chemical groups which will target the detectable construct to particular regions of the body, organ or cellular region or environment. For example, the addition of carbohydrate moieties such as galactose, may cause concentration of the detectable constructs in liver, kidneys and spleen.

Additionally, the detectable compositions of the present invention may comprise an inhibitor of an enzyme, wherein the administration of the inhibitor molecule, separately or as a part of a detectable construct, would create a null space, where the change in the T₂/T₁ ratio would not be measured. For example, a series of MRI measurements may be made, wherein the inhibitor is not present in the first MRI and enzyme activity is detected using a detectable construct and measuring the T₂/T₁ ratio change, and a second MRI is conducted with the inhibitor present, and no change in T₂/T₁ ratio is seen where formally the change was seen.

The expression of enzyme molecules and their associated in vivo inhibitors or substrates is a good indicator as to the type of tissue or its condition. Detectable constructs of the present invention comprise reaction regions which are altered by enzymes which have elevated levels or activity in patients who have inflammatory diseases, infectious disease, cancer, atherosclerosis, thrombosis, myocardial infarction, rheumatoid arthritis, osteoarthritis, endometriosis, periodontal disease, autoimmune disease, and so forth. One class of enzymes is the EC class of enzymes known as Hydrolases (EC 3.1.*.* through EC 3.99.*.*). The enzyme activity sites are carbon-oxygen, carbon-nitrogen, phosphorous-oxygen, carbon-carbon and other bonds which are hydrolytically cleaved by the action of the appropriate enzyme. Other enzyme activity sites are phosphorous-oxygen bonds, which are hydrolysed by enzymes known as phosphatases (EC.3.1.3.*) (Class, Hydrolase; subclass, esterase; sub-subclass, phosphomonoesterase). Specific examples of phosphatase enzymes and their common names alkaline phosphatase, alkaline phospho-monoesterase; phosphomono-esterase; glycero-phosphatase, acid phosphatase, acid phosphomono-esterase; phosphomono-esterase; glycero-phosphatase. The clinical relevance of enzymes which act on phosphorous-oxygen sites is exemplified by the case of acid phosphatase, which has elevated levels in prostate cancer patients and has been used extensively in the diagnosis, staging and monitoring of prostate cancer for decades.

Additional detectable construct reaction regions include those which are cleaved by sulfatases (EC 3.1.6.*; Class, Hydrolase; subclass, esterase; sub-subclass, sulfatase), enzymes which cleave sulfur-oxygen bonds. Steroid sulfatase activity is particularly high in breast tumors, and plays a role in regulating the formation of estrogens within tumors. Specific examples estrone sulfatase, estrone sulfo-transferase, Steroid sulfotransferase, Steryl-sulfatase, Arylsulfatase Sulfatase, N-acetylgalactosamine-6-sulfatase, Disulfoglucosamine-6-sulfatase, Glucuronate-2-sulfatase, Choline-sulfatase, Cerebroside-sulfatase, Chondro-4-sulfatase, Chondro-6-sulfatase, N-acetylgalactosamine-4-sulfatase, Iduronate-2-sulfatase, Monomethyl-sulfatase, D-lactate-2-sulfatase.

Other detectable construct reaction regions include those which are carbon-nitrogen peptide bonds which are hydrolyzed by a subclass of hydrolase enzymes known as proteinases (EC 3.4.*.*). These enzymes hydrolyze an amide bond to form two cleavage products, an amine and a carboxylic acid. Reaction regions that are hydrolyzed by serine proteases (EC 3.4.21.*; Class, Hydrolase; subclass, peptidase, sub-subclass, serine endopeptidase) are included in the present invention. Serine protease activity has been linked to primary breast cancer, tumor progression that leads to metastasis in breast cancer, the activation of coagulation in patients with lung cancer, pancreatic cancer, severe pancreatitis, and prostate cancer. Additional detectable construct reaction regions include those which are useful as diagnostic agents for prostate cancer such as one which is altered by prostate-specific antigen (PSA), a serine protease glycoprotein (30-34 kDa) produced exclusively by prostatic tissue. PSA exhibits enzymatic activity typical of peptidases chymotrypsin and trypsin, and its physiological substrate appears to be high-molecular-mass seminal vesicle protein (HMM-SV-protein). PSA is useful for monitoring therapy, particularly prostatectomy because its presence is decreased to nearly zero following removal of the prostate. A slow rise in PSA following prostatectomy indicates that either not all of the prostate is removed or that lymph node metastases are present and producing the antigen. The concentration of PSA is also proportional to tumor burden or malignant potential and changes quickly in response to therapy. Examples of serine proteases Prostate-specific Semenogelase; antigen PSA; gamma-seminoprotein seminin, Leukocyte Elastase, Lysosomal elastase; Neutrophil; elastase; Bone marrow serine protease; Medullasin; Pancreatic Elastase, Pancreato-peptidase E; Pancreatic elastase I, Myeloblastin Proteinase 3, and Wegener's autoantigen.

Detectable construct reaction regions include those which are altered by matrix metalloproteinases (MMPs) (EC 3.4.24.*, subclass, peptidase; sub-subclass metalloendopeptidase), enzymes which exhibit high bioactivity in the extracellular space, a tissue compartment which is easily accessible to contrast agents. Furthermore, MMP activity is altered by many diseases. To varying degrees, members of the MMP family are linked to the following diseases: cancer (especially in the degradation of extracellular matrix prior to metastases), atherosclerosis (especially in the degradation of the fibrous cap of atherosclerotic plaque leading to rupture, thrombosis, and myocardial infarction or unstable angina), rheumatoid arthritis and osteoarthritis (destruction of cartilage aggrecan and collagen), periodontal disease, inflammation, autoimmune disease, organ transplant rejection, ulcerations (corneal, epidermal, and gastric), scieroderma, epidermolysis bullosa, endometriosis, kidney disease, and bone disease. Specific metalloproteinase enzymes include Matrilysin MMP-7; Matrin; Uterine metallo-endopeptidase; PUMP-1, Interstitial MMP-1; collagenase Vertebrate collagenase; Fibroblast collagenase, Stromelysin-1 MMP-3; Transin; Proteoglycanase. Stromelysin-2 MMP-10; Transin-2, Gelatinase MMP-2; 72-kDa gelatinase; Type IV collagenase; Pseudolysin Pseudomonas in elastase; Pseudomonas aeruginosa-neutral metalloproteinase, Neutrophil MMP-8 collagenase, Gelatinase B MMP-9; 92-kDa gelatinase; Type V collagenase; 92-kDa type IV-collagenase; Macrophage gelatinase, Deuterolysin Penicillium Rogqueforti-protease II; Microbial neutral-proteinase II; and acid metalloproteinase. For example, when the targeted bioactivity is the enzymatic activity expressed by MMP-1, a matrix metalloproteinase which is elevated in certain inflammatory diseases, detectable construct reaction region may comprise a carbon-nitrogen amide bond linking the amino acids glycine (Gly) and isoleucine (Ile). Other enzymes, such as those categorized as esterases (EC 3.1.*.*) or ether hydrolases (EC 3.3.*.*) can also be detected using the present invention.

The present invention also comprises detectable constructs comprising reaction regions wherein the reaction region comprises a nucleic acid. The nucleic acid may be single-stranded or double stranded, and includes nucleic acid analogs such as peptide nucleic acids and other well-known modifications of the ribose-phosphate backbone, such as phosphorthioates, phosphoramidates, morpholino structures, etc. The target bioactivity site can be a substantially complementary nucleic acid or a nucleic acid binding moiety, such as a protein.

The present invention also comprises detectable constructs comprising reaction regions wherein the reaction region comprises a photocleavable moiety. That is, upon exposure to a certain wavelength of light, the reaction region is cleaved. This embodiment has particular use in developmental biology fields (cell lineage, neuronal development, etc.), where the ability to follow the fates of particular cells is desirable. Suitable photocleavable moieties are similar to “caged” reagents which are cleaved upon exposure to light. A particularly preferred class of photocleavable moieties are the O-nitrobenzylic compounds, which can be synthetically incorporated into a blocking moiety via an ether, thioether, ester (including phosphate esters), amine or similar linkage to a heteroatom (particularly oxygen, nitrogen or sulfur). Also of use are benzoin-based photocleavable moieties. Suitable photocleavable moieties are known in the art.

Detectable agents of the present invention comprise elements or molecules that can be detected. Metals are commonly used detectable agents. The selection of the metal atom effects the measured relaxivity of the complex. Paramagnetic metal ions, as a result of their unpaired electrons, act as potent relaxation enhancement agents. They decrease the T₁ and T₂ relaxation times of nearby (r₆ dependence, does not always have to r₆ dependence) spins. Some paramagnetic ions decrease the T₁ without causing substantial linebroadening (e.g. gadolinium (III), (Gd³⁺)), while others induce linebroadening (e.g. superparamagnetic iron oxide). The mechanism of T₁ relaxation is generally through space dipole-dipole interaction between the unpaired electrons of the paramagnet (the metal atom with an unpaired electron) and water molecules that are in the metal's inner coordination sphere. These water molecules are in rapid exchange with the bulk water (water molecules that are not “bound” to the metal atom) and thereby metal can influence the bulk water magnetization properties. For example, regions associated with a Gd³⁺ ion (near-by water molecules) appear bright in an MR image where the normal aqueous solution appears as dark background if the time between successive scans in the experiment is short (i.e. T₁ weighted image). Localized T₂ shortening caused by superparamagnetic particles is believed to be due to the local magnetic field inhomogeneities associated with the large magnetic moments of these particles. Regions associated with a superparamagnetic iron oxide particle appear dark in an MR image where the normal aqueous solution appears as high intensity background if the echo time (TE) in the spin-echo pulse sequence experiment is long (i.e. T₂-weighted image). The lanthanide atom Gd³⁺ is by the far the most frequently chosen metal atom for MRI contrast agents because it has a very high magnetic moment (u²=63BM²), and a symmetric electronic ground state, (S⁸). Transition metals such as high spin Mn(II) and Fe(III) are contemplated by the present invention due to their high magnetic moments.

The detectable agents of the present invention comprise a molecule or element that is detected by the imaging technique employed. The metal ion complexes of the present invention generally comprise a paramagnetic metal ion bound to a chelator molecule. By “paramagnetic metal ion”, “paramagnetic ion” or “metal ion” herein is meant a metal ion which is magnetized parallel or antiparallel to a magnetic field to an extent proportional to the field. Generally, these are metal ions which have unpaired electrons; this is a term understood in the art. Examples of suitable paramagnetic metal ions, include, but are not limited to, gadolinium III (Gd⁺³ or Gd(III)), iron III (Fe⁺³ or Fe(III)), manganese II (Mn⁺² or Mn(II)), yttrium III (Y+³ or Y(III)), dysprosium (Dy⁺³ or Dy(III)), and chromium (Cr(III) or Cr⁺³). Imaging techniques may also detect detectable agents including an organic molecule, metal ion, salt or chelate, cluster, particle (particularly iron particle), or labeled peptide, protein, or polymer. For ultraviolet/visible/infrared/fluorescence light (optical) imaging, the detectable agent may also be any organic or inorganic dye. Particularly useful inorganic dyes include luminescent metal complexes, such as those of Eu(III), Tb(III) and other lanthanide ions (atomic numbers 57-71). See W. Dew. Horrocks & M. Albin, Progr. Inora. Chem. (1984), 31, pp. 1-104. Other detectable agents may include a pharmaceutically acceptable metal chelate compound of one or more cyclic or acyclic organic chelating agents complexed to one or more metal ions. Metal ions preferred for optical imaging include those with atomic numbers 13, 21-34, 39-42, 44-50, or 57-83. Paramagnetic metal ions preferred for MRI include those with atomic numbers 21-29, 42, 44, or 57-83.

In the detectable constructs of the present invention, the metal chelate detectable agent should not dissociate to any significant degree during the passage of the detectable construct through the body, including a tissue where it may undergo biomodification. Significant release of free metal ions can result in large MRI or optical signal alterations, and may also be accompanied by toxicity, which would only be acceptable in pathological tissues. It is preferred that bioactivation not significantly compromise the stability of the detectable agent region so that the metal complex can remain intact and be excreted.

Exemplary detectable agents include, but are not limited to, iron enterobactin, iron MECAMS, gadolinium diethylenetriamine pentaacetic acid (“DTPA”), gadolinium (1,4,7,10-tetraazacyclotetradecene 1,4,7,10-tetracetic acid (“DOTA”), gadolinium DTPA-BMA, and gadolinium EDTA, iron particle or metal chelate of high magnetic susceptibility, particularly chelates of Dy, Gd, or Ho, or other metals that can alter the MRI signal intensity of tissue by creating microscopic magnetic susceptibility gradients, or iron particle or metal chelate that can shift the resonance frequency of water protons or other imaging or spectroscopic nuclei, including protons, P-31, C-13, Na-23, or F-19 on nearby atoms, and paramagnetic metals including Gd(III), Fe(III), Mn(II) and Mn(III), Cr(III), Cu(II), Dy(III), Tb(III), Ho(III), Er(III) and Eu(III).

The compositions of the present invention may also comprise detectable constructs that comprise a chelator or chelator-like molecule, which is used for attaching or binding the metal ion with the detectable construct molecule. Molecules are known in the art that can serve this function and can be chosen based on factors such as the stability of chelate-metal complexes, and enthalpy and entropy effects such as number, charge and basicity of coordinating groups, ligand field and conformational effects. The chelate molecule can be modified to provide desired characteristics to the detectable construct. For example, the presence of a methyl group on a chelate molecule can have an effect on clearance rate. The addition of a bromine group may cause a detectable construct to shift from a purely extracellular role to an agent that collects in hepatocytes.

Examples of chelator molecules of the present invention are known in the art and include, but are not limited to, the following. Diethylenetriaminepentaacetic (DTPA) chelates lanthanide ions. The water soluble Gd(DTPA)²-chelate is stable, nontoxic, and is a widely used contrast enhancement agents in experimental and clinical imaging research. Other chelators include diethylenetriaminepentaacetic (DTPA), 1,4,7,10-tetraazacyclododecane′-N,N-′N″,N′″-tetracetic acid (DOTA), and derivatives thereof. See U.S. Pat. Nos. 5,155,215, 5,087,440, 5,219,553, 5,188,816, 4,885,363, 5,358,704, and 5,262,532.

In general, the organic chelator molecule should be physiologically compatible. The molecular size of the chelator molecule should be compatible with the size of the paramagnetic metal. Known chelator molecules include DTPA, DOTA, DTPA-BMA or HP-DO3A.

Modification of chelator molecules is contemplated by the present invention. There are a variety of factors which influence the choice and stability of the chelate metal ion complex, including enthalpy and entropy effects (e.g. number, charge and basicity of coordinating groups, ligand field and conformational effects). In general, the chelator has a number of coordination sites containing coordination atoms which bind the metal ion. The number of coordination sites, and thus the structure of the chelator, depends on the metal ion. There are a large number of known macrocyclic chelators or ligands which are used to chelate lanthanide and paramagnetic ions. See for example, Alexander, Chem. Rev. 95:273-342 (1995) and Jackels, Pharm. Med. Imag, Section III, Chap. 20, p 645 (1990), expressly incorporated herein by reference, which describes a large number of macrocyclic chelators and their synthesis. Similarly, there are a number of patents which describe suitable chelators for use in the invention, including U.S. Pat. Nos. 5,155,215, 5,087,440, 5,219,553, 5,188,816, 4,885,363, 5,358,704, and 5,262,532, all of which are expressly incorporated by reference. Substituted or modified chelators are also taught in many references. For example suitable substitution groups for DOTA (1,4,7,10-tetraazacyclododecane-N,N′,N″, N′″-tetracetic acid) (DOTA) or DOTA-type compounds are taught inin U.S. Pat. Nos. 5,262,532, 4,885,363, and 5,358,704. These groups include hydrogen, alkyl groups including substituted alkyl groups and heteroalkyl groups, aryl groups including substituted aryl and heteroaryl groups, phosphorus moieties, and blocking moieties.

By “alkyl group” or grammatical equivalents herein is meant a straight or branched chain alkyl group, with straight chain alkyl groups being preferred. If branched, it may be branched at one or more positions, and unless specified, at any position. Also included within the definition of alkyl are heteroalkyl groups, wherein the heteroatom is selected from nitrogen, oxygen, phosphorus, sulfur and silicon. Also included within the definition of an alkyl group are cycloalkyl groups such as C5 and C6 rings, and heterocycloalkyl. Additional suitable heterocyclic substituted rings are depicted in U.S. Pat. No. 5,087,440, expressly incorporated by reference. In some embodiments, two adjacent R groups may be bonded together to form ring structures together with the carbon atoms of the chelator, such as is described in U.S. Pat. No. 5,358,704, expressly incorporated by reference. These ring structures may be similarly substituted. The alkyl group may range from about 1 to 20 carbon atoms (C1-C20), with a preferred embodiment utilizing from about 1 to about 10 carbon atoms (C1-C10), with about C1 through about C5 being preferred. However, in some embodiments, the alkyl group may be larger, for example when the alkyl group is the coordination site barrier.

By “alkyl amine” or grammatical equivalents herein is meant an alkyl group as defined above, substituted with an amine group at any position. In addition, the alkyl amine may have other substitution groups, as outlined above for alkyl group. The amine may be primary (—NH₂R), secondary (—NHR₂), or tertiary (—NR₃). When the amine is a secondary or tertiary amine, suitable R groups are alkyl groups as defined above.

By “aryl group” or grammatical equivalents herein is meant aromatic aryl rings such as phenyl, heterocyclic aromatic rings such as pyridine, furan, thiophene, pyrrole, indole and purine, and heterocyclic rings with nitrogen, oxygen, sulfur or phosphorus. Included within the definition of “alkyl” and “aryl” are substituted alkyl and aryl groups. That is, the alkyl and aryl groups may be substituted, with one or more substitution groups. For example, a phenyl group may be a substituted phenyl group. Suitable substitution groups include, but are not limited to, halogens such as chlorine, bromine and fluorine, amines, hydroxy groups, carboxylic acids, nitro groups, carbonyl and other alkyl and aryl groups as defined herein. Thus, arylalkyl and hydroxyalkyl groups are also suitable for use in the invention. Preferred substitution groups include alkyl amines and alkyl hydroxy. By “phosphorous moieties” herein is meant moieties containing phosphorus-containing groups. The phosphorus may be an alkyl phosphorus; for example, DOTEP utilizes ethylphosphorus as a substitution group on DOTA. DOTEP is 1,4,7,10-tetraazacyclododecane-N,N′,N″,N′″-tetraeth-ylphosphorus (DOTEP) and includes substituted DOTEP, as taught in U.S. Pat. No. 5,188,816. DPTA is also a chelator molecule contemplated by the present invention which includes substituted DPTA, and such substitutions are taught U.S. Pat. No. 5,087,440. DOTEP and DPTA may have similar substitution groups as outlined above. Other suitable chelators are described in U.S. Pat. Nos. 5,155,215, 5,087,440, 5,219,553, 5,188,816, 4,885,363, 5,358,704, and 5,262,532.

When the paramagnetic ion is Fe(III), suitable chelators for Fe(III) ions are well known in the art, see for example U.S. Pat. Nos. 4,885,363, 5,358,704, and 5,262,532, all which describe chelators suitable for Fe(III). When the paramagnetic ion is Mn(II) (Mn⁺²), suitable chelators for Mn(II) ions are well known in the art; see for example U.S. Pat. Nos. 4,885,363, 5,358,704, and 5,262,532. When the paramagnetic ion is Y(III), suitable chelators for Y(III) ions include, but are not limited to, DOTA and DPTA and derivatives thereof and those chelators described in U.S. Pat. No. 4,885,363. When the paramagnetic ion is Dy⁺³ (Dy(III)), suitable chelators are known in the art, as above.

In general, chelators of the invention may be modified so that the chelator molecule can be covalently bonded to other regions of the detectable construct. Such modifications may include one or more substitution groups that serve as functional groups for chemical attachment. Suitable functional groups include, but are not limited to, amines (preferably primary amines), carboxy groups, and thiols (including SPDP, alkyl and aryl halides, maleimides, alpha-haloacetyls, and pyridyl disulfides) are useful as functional groups that can allow attachment.

The present invention comprises detectable constructs comprising a linking region. In some embodiments, the linking region may be a polymer. Polymers which may be used in detectable constructs of the present invention include, but are not limited to, dextrans, styrene polymers, polyethylene and derivatives, polyanions including, but not limited to, polymers of heparin, polygalacturonic acid, mucin, nucleic acids and their analogs including those with modified ribose-phosphate backbones, polypeptides, polyglutamate, polyaspartate, carboxylic acid, phosphoric acid, and sulfonic acid derivatives of synthetic polymers; and polycations, including but not limited to, synthetic polycations based on acrylamide and 2-acrylamido-2-methylpropanetrimethylamine, poly(N-ethyl-4-vinylpyridine) or similar quarternized polypyridine, diethylaminoethyl polymers and dextran conjugates, polymyxin B sulfate, lipopolyamines, poly(allylamines), poly(dimethyldiallylammonium chloride), polyethyleneimine, polybrene, spermine, spermidine, protamine, the histone polypeptides, polylysine, polyarginine and polyornithine; and mixtures, derivatives and combinations of these are contemplated by the present invention. Linear and branched polymers may be used in the detectable constructs of the present invention.

The size of the polymer may vary. For example, it is known that some nucleic acid vectors can deliver genes up to 100 kilobases in length, and artificial chromosomes (megabases) have been delivered to yeast. Therefore, there is no general size limit to the polymer. The polymer may be from about 10 to about 50,000 monomer units, from about 2000 to about 5000, and from about 3 to about 25. Alternatively, the polymer may be from 500MW to 10,000,000 MW.

An aspect of the present invention is shown in FIG. 14 of a detectable construct for imaging bioactivity, and a method of use of detectable constructs of the present invention can be referred to herein as T₂/T₁ signal quenching. This method uses the opposing effects that T₂ and T₁ contrast agents have on MRI signal intensity to detect bioactivity. The T₂/T₁ quenching method can be used to image many types of bioactivity, for example, to image the location enzymes through the bioactivity of the enzymes on the detectable constructs.

A T₂/T₁ quencher is a detectable construct that comprises a T₁ detectable agent, or contrast agent, (gadolinium) and a T₂ detectable agent, or contrast agent, (dysprosium) (1), shown in FIG. 14. The detectable construct has a reaction region, for example, an enzyme cleavable region and, in this instance, is joined directly to the T₂ contrast agent. The detectable construct also has a linking region. T₁ contrast agents increase MRI signal intensity, whereas T₂ contrast agents decrease MRI signal intensity. This relationship is quantitatively described in the spin-echo signal's dependence on relaxation times as given in the following equation (Haacke 1999):

$$( 1 - {ⅇ}^{\frac{TR}{{T}_{1}}} ) {ⅇ}^{\frac{TE}{{T}_{2}}}$$

Thus a T₂/T₁ quenching detectable construct has a weak effect on MRI signal intensity, because the dysprosium quenches the effects of the gadolinium. However, after cleavage of the T₂/T₁ quenching detectable construct by enzyme activity, a low molecular weight dysprosium detectable construct remainder and a polymeric gadolinium detectable construct remainder (2) are generated (see FIG. 14). The low molecular weight dysprosium detectable construct remainder has a higher translational diffusion coefficient and lower rotational correlation time than the intact T₂/T₁ quenching detectable construct and the polymeric gadolinium detectable construct remainder. Additionally, a detectable construct remainder may have an altered water exchange rate, that is different from that of the intact, or not acted upon, detectable construct which allows for a measurable change to be detected. The cleaved dysprosium detectable construct remainder diffuses away from the polymeric gadolinium detectable construct remainder and also has a reduced effect on T₂. Thus enzymatic cleavage of the T₂/T₁ quenching detectable construct eliminates the quenching effects of the dysprosium, resulting in an increase in MRI signal intensity and the detection of the enzyme through its bioactivity. The method is equally effective is detectable constructs where the reaction region is more closely joined or directly joined to the T₁ contrast agent.

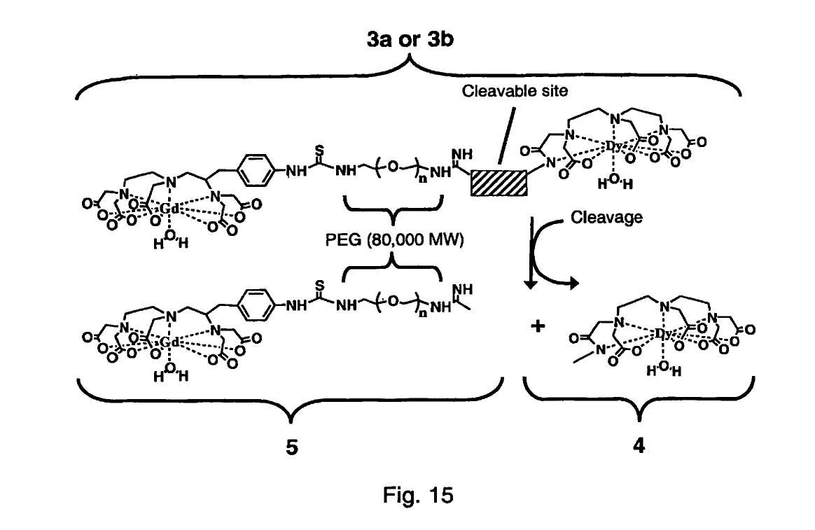

Another example of a detectable construct of the present invention is shown in FIG. 15, wherein the detectable construct comprises a first detectable agent, comprising a metal ion and a chelator, covalently bonded to a linking region comprising PEG(polyethylene glycol) MW 80 kD, which is covalently bonded to a reaction region that is a substrate that can be cleaved by an enzyme or chemical process, which is covalently bonded to a second detectable agent comprising a chelator and a metal ion. In this particular example, the first detectable agent is Gadolinium associated with the chelator DTPA, and the second detectable agent is Dysprosium associated with the chelator DTPA. This detectable construct can be used in a method based upon the counteracting effects of T₁ and T₂ contrast agents on MRI signal intensity. Gadolinium is the T₁ contrast agent and dysprosium is the T₂ contrast agent. The dysprosium detectable agent is connected to the PEG chain through a cleavable linkage, such as a disulfide bond or an enzyme cleavable peptide linkage. Cleavage of 3a generates a low molecular weight dysprosium detectable construct remainder (4) and a high molecular weight PEG gadolinium detectable construct remainder. Though not wishing to be bound by any particular theory, it is believed that the low molecular weight dysprosium detectable construct remainder generated from cleavage of the T₂/T₁ quenching detectable construct will diffuse away from the high molecular weight PEG-gadolinium detectable construct remainder, leading to an increase in tissue T₂ and MRI signal intensity. This belief considers estimates of the diffusion coefficients of the cleaved dysprosium detectable construct remainder (4) and the PEG-gadolinium detectable construct remainder (5). For example, in water a PEG of 80 kD has a diffusion coefficient D_{water}=2.2×10⁻⁷ cm²/s (Huang 2002), whereas for DTPA-dysprosium D_{water}=3.8×10⁻⁶cm²/s (Gordon 1999), which is 17 times greater than the diffusion coefficient of the PEG 80 kD. Furthermore, in hydrogel environments, such as the tissue extracellular matrix, the diffusion coefficient of the PEG-gadolinium detectable construct remainder is reduced more than the dysprosium detectable construct remainder, due to the porous nature of hydrogels (Pluen 1999 and Ramanujan 2002).

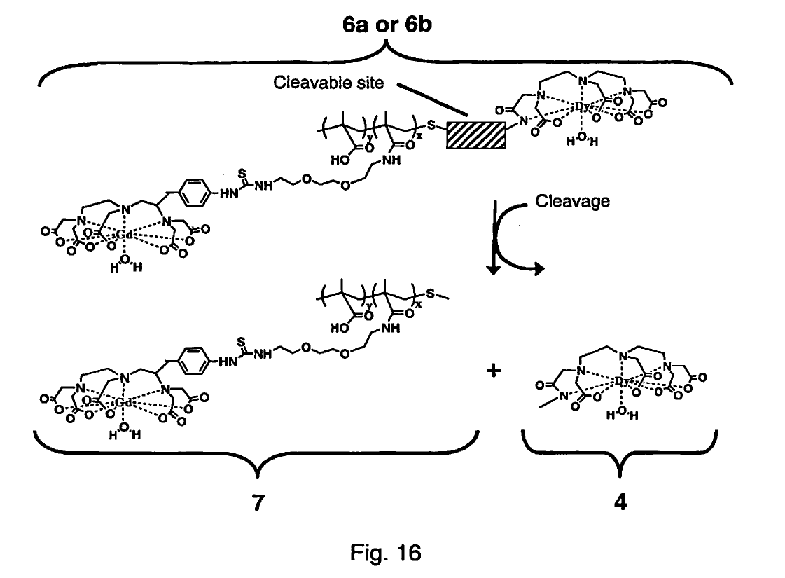

Another example of a detectable construct of the present invention is shown in FIG. 16, wherein the detectable construct comprises a first detectable agent comprising a metal ion and a chelator, covalently bonded to a linking region that is rotationally hindered, as shown with a rotationally hindered poly(methacrylic acid) chain (PMAA), which is covalently bonded to a reaction region that is a substrate that can be cleaved by an enzyme or chemical process, which is covalently bonded to a second detectable agent comprising a chelator and a metal ion. In this particular example, the first detectable agent is Gadolinium associated with the chelator DTPA, and the second detectable agent is Dysprosium associated with the chelator DTPA. This detectable construct can be used in a method based upon the counteracting effects of T₁ and T₂ contrast agents on MRI signal intensity. Gadolinium is the T₁ contrast agent and dysprosium is the T₂ contrast agent. The dysprosium detectable agent is connected to the rotationally hindered poly(methacrylic acid) chain (PMAA) chain through a cleavable linkage, such as a disulfide bond or an enzyme cleavable peptide linkage. The T₂ effects of dysprosium are proportional to its rotational correlation time. For example, dysprosium complexed to BSA has a rotational correlation time (τᵣ) of approximately 7000×10⁻¹²s and an R₂ of 2.0 mM⁻¹s⁻¹, whereas dysprosium-diethylenetriaminepentaacetic (DTPA) has a τᵣ of 58.0×10⁻¹²s and an R₂ of 0.5 mM⁻¹s⁻¹ (at 4.7 Tesla) (Caravan 2001).

The quenching detectable construct of FIG. 16 uses this sensitivity to rotational correlation to generate a change in tissue T₂ with enzyme cleavage. The detectable construct of either 6a or 6b has a rotational correlation time comparable to PMMA, which has a τᵣ of 5000×10⁻¹²s (for 58 kD Mn) (Pilar 1991a and 1991b). However after cleavage of the reaction region (herein 6a or 6b), the released dysprosium detectable construct remainder will have a rotational correlation time comparable to a low molecular weight dysprosium ion and its chelator molecule, which has a τᵣ of 58.0×10⁻¹²s. This change in rotational correlation time results in a decrease in R₂ and thus an increase in tissue T₂ and signal intensity. One aspect of using the T₂/T₁ quenching detectable construct of FIG. 16 is that enzyme cleavage causes a rapid, if not immediate, decrease in dysprosium's R₂ and result in a rapid, if not immediate, increase in MRI signal intensity, whereas in the T₂/T₁ quenching detectable construct of FIG. 15, the cleaved dysprosium detectable construct remainder has to diffuse away before the increase in signal intensity is generated.

It is currently theorized that the R₂ effect of dysprosium is proportional to the square of the magnetic field strength. At low field strengths, such as 1.5 Tesla, the R₂ of dysprosium chelates are, in general, lower than the R₂ of gadolinium chelates. The methods and compositions taught herein where a T₁ and a T₂ detectable agents are provided, and the T₂/T₁ quenching and ratio is determined, are effective at various field strengths, and can be designed to be used at low to high field strengths including from about 0.001 Tesla to about 40.0 Tesla; from about 1.0 Tesla to about 25.0 Tesla; from about 1.0 Tesla to about 35.0 Tesla; from about 20 Tesla to about 40.0 Tesla; from about 2.0 Tesla to about 15.0 Tesla; from about 3.0 Tesla to about 8.0 Tesla; from about 4 Tesla to about 8.0 Tesla; from about 3.0 Tesla to about 15.0 Tesla, from about 0.01 Tesla to 25 Tesla, from about 0.1 Tesla to about 30 Tesla and from about 0.001 Tesla to about 10 Tesla. At low field strengths only a small change in tissue T₂ will occur from enzyme cleavage, due to the dominating effects of the gadolinium R₂. The maximum field strength of currently used clinical MRI machines is between 4.0 and 7.0 Tesla. However, the T₂/T₁ quenching detectable constructs are designed to be used at clinically relevant field strengths by modifying the detectable agent, for example, by modifying the dysprosium detectable agent so that its R₂ is comparable to that of the gadolinium detectable agent at low field strengths. For example, this is accomplished by increasing the dysprosium detectable construct remainder rotational correlation time and decreasing its water exchange rate. The R₂ of these modified dysprosium detectable agents is between 3.0-5.0 mM⁻¹s⁻¹ at field strengths in the range of 4.7 and 7 Tesla, and is comparable to the R₂ of gadolinium chelates (Vander 2002 and Caravan 2001). The same considerations are applicable to modifications for the Gd detectable agent, or other detectable agents useful for the present invention. Modifications can be made to any region of the detectable construct that aid in the image produced.

An example of the present invention comprises methods for administering detectable compositions comprising subcompositions comprising a soluble detectable construct comprising a polymeric linking region and a T2 detectable agent (2 kD-500 kD), and also comprising an enzyme cleavable reaction region. Cleavage of the detectable construct by enzymes causes a reduction in the R2 of the T2 detectable construct remainder, which leads to an increase in T2 and the detection of the enzyme activity and location of the enzyme. Furthermore, the accuracy of enzyme quantitation is assured by co-delivering, either simultaneously or sequentially, a subcomposition comprising a soluble detectable construct comprising a polymeric linking region and a T1 contrast agent (2 kD-500 kD), which does not comprise the same reaction region or comprises no reaction region.

The present invention also comprises detectable compositions and methods for making and using such detectable compositions wherein the detectable compositions comprise at least one detectable construct wherein the detectable construct comprises a hydrogel. Methods of detecting bioactivity comprise administering the detectable construct comprising a hydrogel. The detectable construct comprising a hydrogel comprises two different detectable agents for example, a T₂ detectable agent and a T₁ detectable agent, wherein one of the detectable agents is connected to the hydrogel through a reaction region, and the other detectable agent is connected to the hydrogel through a different reaction region or no reaction region. The detectable construct may also comprise linking regions or other regions disclosed herein, and the modifications and other alternatives discussed herein. For example, a detectable construct may comprise a detectable construct comprising a hydrogel wherein a T₂ detectable agent is connected to the hydrogel through a first reaction region, for example, an enzyme cleavable reaction region, and the T₁ detectable agent is immobilized and bound to the hydrogel directly or by a region that is different from the first reaction region bound to the first detectable agent. Exposure of this gel to the particular enzyme for which the first reaction region is the substrate or provides some other interaction with the target enzyme, leads to alteration of the first reaction and change in the measured T₂, for example, cleavage and release of the T₂ agent, which results in an increase in T₂ of the hydrogels, which leads to the detection of the enzyme. Furthermore, by using the T₁ agent as an internal reference, the ratio of cleaved versus intact T₂ agent can be determined. An aspect of the detectable construct comprising a hydrogel comprises a hydrogel covalently bonded to a chelator molecule, for example, DOTA, with which a paramagnetic ion is associated, for example, Gd. Multiple sites in the hydrogel are covalently bonded to DOTA and its Gd ion. At separate sites in the hydrogel, at least one other detectable agent is covalently bonded to a region that is covalently bonded to the hydrogel. For example, a detectable agent comprising DOTA and Dy may be covalently bonded to a reaction region that is covalently bonded to the hydrogel. For example, a detectable agent comprising DOTA and Dy may be covalently bonded to a reaction region that is covalently bonded to a linking region that is covalently bonded to the hydrogel. Alternatively, a detectable agent comprising DOTA and Dy may be covalently bonded to a linking region that is covalently bonded to a reaction region that is covalently bonded to the hydrogel.

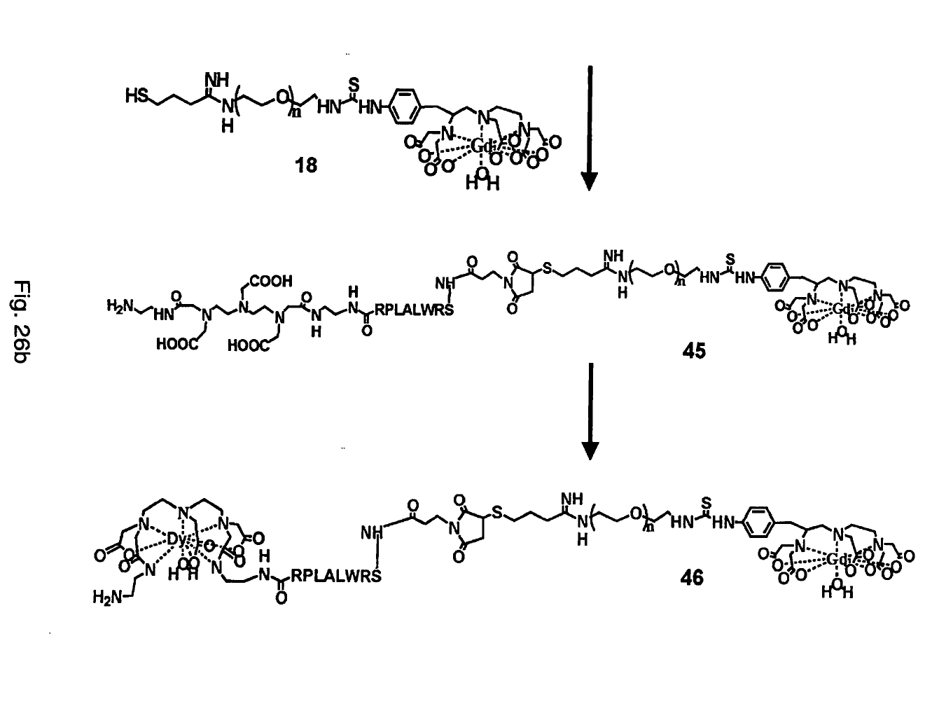

The present invention also comprises detectable compositions and methods for making and using such detectable compositions wherein the detectable compositions comprise at least one detectable construct wherein the detectable construct comprises nanometer-sized micelles that have one or more detectable agents such as dysprosium encapsulated in the core space of the micelle, and a second detectable agent, such as gadolinium, on the periphery of the micelle. Such compositions can be used in methods of imaging bioactivity including those discussed herein. (see FIG. 26). For example, Dysprosium influences MRI contrast by lowering the T₂ of water, which decreases MRI signal intensity. Importantly, dysprosium's effects on water T₂ are very sensitive to the concentration gradient of dsyprosium. This is due to dysprosium's high magnetic susceptibility and Curie spin mechanism of relaxation. Dysprosium's sensitivity to concentration gradients is dramatic, for example dysprosium encapsulated in red blood cells is 40 times more effective at lowering water T₂ than free dysprosium in solution, due to the concentration gradient generated by sequestering dysprosium in the red blood cells. Micelles containing detectable agents such as dysprosium encapsulated within the core of the micelle have a higher effect on water T₂ than that same quantity of dysprosium dispersed evenly through solution. In this method and composition, the micelles initially have a detectable agent such as dysprosium encapsulated in the core and have a high effect on water T₂ because of the concentration gradient generated by the micelle. However, in the presence of the target bioactivity, the peptide cross-linked micelles are degraded and the encapsulated detectable agent, such as dysprosium, is released, which dissipates the dysprosium concentration gradient, resulting in an increase in MRI signal intensity and the detection of the bioactivity and consequently, the enzyme. Gadolinium is attached to the periphery of the micelles, to act as internal standard.

The present invention contemplates the micellar compositions and micelles taught herein and one skilled in the art could apply the aspects of the detectable constructs disclosed herein to include detectable constructs wherein a micelle is intended, and for methods in addition to detection methods.

The medical benefits of diagnosing cancer at an early stage are undisputed. Although significant progress has been made in both imaging equipment and molecular imaging technology, there remains an unmet medical need for definitive diagnostics that can detect cancer at an early stage and reliably pinpoint its location. T₂/T₁ signal quenching as disclosed herein represents a powerful approach toward developing a family of new MRI contrast agents, designed to detect enzymes secreted by cancer cells during the earliest stages of cancer formation, thereby permitting early treatment and dramatically improved disease prognosis. The association of certain enzymes with cancer activity is well established from data generated from animal models of cancers and human tissue biopsies.

At present, tumors can only be detected after they reach mm-cm dimensions. The method of signal quenching, because of its ability to detect enzyme activity in vivo, has the potential to image tumors when they are at the non-μm scale, which is 2-3 orders of magnitude better than conventional diagnostics. This substantial improvement in imaging capability can be expected to lead to a more effective surveillance in high risk patients, earlier definitive diagnosis, and much less severe and more effective treatment modalities. In general, the signal quenching methods taught herein, comprises use of at least two different detectable agents, and measuring the T₂/T₁ ratio for image creation. An intact T₂/T₁ quenching detectable construct will have a weak effect on MRI signal intensity when one detectable agent quenches the effects of the second detectable agent, such as in a dysprosium detectable agent quenches the effects of the gadolinium detectable agent. However, alteration of the relative position of one detectable agent to the other detectable agent, such as by cleavage of a reaction region by enzymes, will generate a measurable difference in the signal generated after the alteration compared to the initial measurement, for example a low molecular weight dysprosium detectable construct remainder will have an increased translational diffusion coefficient and decreased rotational correlation time. The changes in these two parameters will reduce the quenching effects of the dysprosium detectable agent on the gadolinium detectable agent, resulting in an increase in MRI signal intensity and the detection of the bioactivity and the enzyme. For example, for the detectable construct of FIG. 15, the large PEG-DTPA-Gd detectable construct remainder should have a diffusion coefficient in water of approximately 2.2×10⁷ cm²/s (Huang 2002), whereas the smaller DTPA-Dy detectable construct remainder should have a diffusion coefficient of approximately 3.8×10⁻⁶ cm²/s (Gordon 1999), a roughly 17-fold difference. In a porous tissue environment, the DTPA-Dy detectable construct remainder will diffuse more rapidly than the large PEG-DTPA-Gd detectable construct remainder from the site of cleavage, thus reducing the Dy-Gd quenching effect. The DTPA-Dy detectable construct remainder should also have a dramatically reduced rotational correlation time in comparison to the whole detectable construct because of its lower molecular weight, further enhancing signal intensity.

At present, technologies do not exist that can image enzyme activity in vivo in humans or animals. Signal quenching methods allow for a new methodology for imaging any type of enzyme, and locating early-stage disease processes before they become symptomatic. For example, proteolytic enzymes, such as cathepsins and matrix metalloproteinases, are overexpressed and secreted by cancer cells during the earliest stages of tumor growth and metastasis. These enzymes are excellent targets for early diagnosis. Signal quenching methods can quantitatively determine enzyme concentrations in vivo through ratiometric measurements. For example, at the local site where enzyme cleavage of the detectable construct is taking place, measurement of the “tissue T₁” indicates the total concentration of gadolinium present in the tissue (representing the cleaved and intact detectable construct), and measurement of the “tissue T₂” indicates the concentration of dysprosium present in the tissue (representing just the intact detectable construct). By comparing the ratio of these two concentrations, the percentage of cleaved T₂/T₁ detectable construct remainders can be determined, and with knowledge of enzyme catalytic rates, the enzyme concentrations in vivo can be calculated. Clinically, this method permits sensitive and accurate in vivo measurements related to the extent of cancer formation, its progression over time, and its response to therapy.

Importantly, signal quenching methods are currently the only methodology available for ratiometric imaging by MRI for enzyme detection. Other MRI imaging technologies under development for enzyme detection are based upon singular T₂ or T₁ contrast agents. These technologies cannot quantitatively determine enzyme concentrations in vivo, and also have a much harder time distinguishing between artifacts and real enzyme activity.

Another medical application of T₂/T₁ quenching methods is the early detection of vulnerable plaques in cardiovascular disease. In the progression of atherosclerosis, lipid-rich plaques build up in the arterial wall, in many cases without causing significant blockage of the artery, yet still being susceptible to erosion, rupture, and the triggering of acute coronary events. A problem in treating patients with vulnerable plaques is that it is currently impossible to identify such patients early enough in their disease progression to provide effective therapy. The T₂/T₁ quenching methods of the present invention can detect vulnerable plaques before thrombosis occurs by measuring bioactivity, such as endopeptidase MMP-9, a hallmark of the disease process expressed in large quantities prior to rupture of the vessel wall. Using a detectable construct with a reaction region that is cleavable by MMP-9, administration of detectable compositions and signal quenching methods can be used to image the early stages of the disease process, and its progression and response to therapy.

The present invention comprises compositions and methods of making and using micellar compositions. The micellar compositions comprise one or more micelles comprising amphiphillic molecules, including, but not limited to, block copolymers, that self-assemble to form the one or more micelles. As used herein, a micelle is a water soluble or colloidal structure or aggregate, also called a nanosphere or nanoparticles, composed of one or more amphiphilic molecules. The micelles generally have a single, central and primarily hydrophobic zone or “core” surrounded by a hydrophilic layer or “shell”. Micelles range in size from 5 to about 2000 nanometers, preferably from 10 to 400 nm. Micelles of the present invention are distinguished from and exclude liposomes which are composed of bilayers.

Amphiphilic molecules are those that contain at least one hydrophilic (polar) moiety and at least one hydrophobic (nonpolar) moiety. The micelles of the present invention may be composed of a single monomolecular polymer containing hydrophobic and hydrophilic regions, such as a block copolymer having hydrophobic and hydrophilic blocks. The micelle is self-assembled from one or more amphiphilic molecules where the regions are oriented to provide a primarily hydrophobic interior core and a primarily hydrophilic exterior or shell.

A block copolymer of the present invention may comprise blocks of polymeric molecules, and comprise at least two segments, a core-forming block and a shell-forming block. The shell-forming block of the block copolymer may further comprise side-chain moieties suitable for chemical modification such as grafting of chemical moieties onto the side-chains. The micellar compositions further micelles wherein the block copolymers are cross-linked by a cross-linking molecule such as a peptide, an active agent or a reaction region as defined herein. The cross-linking molecule may be a reaction region when the cross-linking molecule or a portion of the cross-linking molecule is acted on chemically or physically by a biological molecule or chemical process of a living organism, bioactivity. The reaction region is altered by the bioactivity. Targeting the delivery of the micelle is achieved by the reaction or association of the reaction region with a specific bioactivity. For example, the reaction region may be the substrate for an enzyme such that when the micelle encounters the enzyme, the substrate or reaction region is acted on by the enzyme. The reaction region may be a chemically reactive combination of elements, such as a disulfide bond, that when in the appropriate environment, the disulfide bond is broken by the specific bioactivity, the micelle is disrupted and any active agents within the micelle are released. The cross-linking molecule may function both to target and as an active agent. For example, when the cross-linking molecule is a peptide that is an immunogen, the micelle is targeted to cells of the immune system, which then initiates an immune response, such as a Tcell or Bcell response.

The micellar compositions may further comprise micelles having active agents encapsulated, trapped, or in some manner located within the core of the micelle. Active agents include drug molecules, pharmaceuticals, therapeutic agents, nutriceuticals, prodrugs, anticancer drugs, antineoplastic drugs, antifungal drugs, antibacterial drugs, antiviral drugs, cardiac drugs, neurological drugs, drugs of abuse; alkaloids, vitamins, chemical elements, minerals, antibiotics, bioactive peptides, steroids, steroid hormones, polypeptide hormones, interferons, interleukins, narcotics, nucleic acids including sense, antisense, or immunostimulatory oligonucleotides, pesticides and prostaglandins, peptides, proteins, ions, inorganic compounds, immunogens, antigens, antibodies, antibody fragments, adjuvants, imaging compounds, detectable agents and detectable compositions taught herein, and mixtures and combinations of the foregoing entities.

The methods of the present invention comprise detecting bioactivity in a body, tissue, cells, including intracellularly or extracellularly by the administration of an effective amount of a micellar composition comprising micelles comprising a cross-linking molecule and can be use in applications that are in vivo, ex vivo, and in vitro. Methods of the present invention comprise targeted delivery of one or more active agents using micellar compositions comprising micelles having the one or more active agents on the outside or on the inside of the micelle or both, to a human or animal. Methods of the present invention comprise targeted delivery of active agents using micellar compositions of the present invention. Methods also comprise delivery of one or more immunogens in methods of vaccination in a human or an animal, wherein an immunogenic active agent, including but not limited to, a viral, bacterial yeast or other immunogenic or antigenic peptide, an immunostimulatory nucleic acid sequence (ISS), adjuvant, hapten, immunostimulatory chemical, or a combination of one or more of these, such as a peptide and ISS, are delivered to immune competent cell, such as an antigen presenting cell (APC).

An aspect of the present invention comprises a micelle wherein the cross-linking molecule is a peptide, referred to herein as a peptide cross-linked micelle, comprising a block copolymer wherein at least one block of the copolymer comprises pendant reactive side chains, wherein an effective amount of the side chains are covalently attached to cross-linking molecules. An effective amount of the side chains is defined as the number of side chains that are participating in the binding with cross-linking molecules such that the micelle is substantially stabilized. The cross-linking molecule may be a peptide comprising two or more reactive amino acids, three or more reactive amino acids, four or more reactive amino acids, or multiple reactive amino acids. The reactive amino acids may be found anywhere in the peptide structure. For example, a cross-linking molecule can be a peptide having two reactive amino acids at the amino terminus of the peptide and two reactive amino acids at the carboxy terminus of the peptide. This exemplary peptide may form four covalent bonds at four sites with one or more pendant reactive side chains of one or more block copolymer molecules. An aspect of the cross-linking comprises covalent bonds between the peptide and the block copolymer such that the covalent cross-links may comprise a reaction region. For example, the covalently bonding may form a disulfide bond and such a disulfide bond is a reaction region for particular chemical processes in living organisms. An aspect of the invention comprises a reaction region comprising the cross-linking molecule itself, wherein the cross-linking molecule is acted on by chemical or biological processes in the body.

Methods for making a peptide cross-linked micelle further comprise mixing one or more block copolymers of the present invention, such as those having pendant reactive side chains, with an active agent wherein the block copolymer self-assembles into a micelle and in so forming, encapsulates or traps the active agent in the core of the micelle. A further step in the method comprises mixing the block copolymer—active agent micelle with the cross-linking molecule, a peptide. Under appropriate reaction conditions, as would be known to one skilled in the art, the peptide forms covalent bonds with the cross-linking moiety, the pendant reactive side chains, of the block copolymer, thereby cross-linking the block copolymers of the micelle.

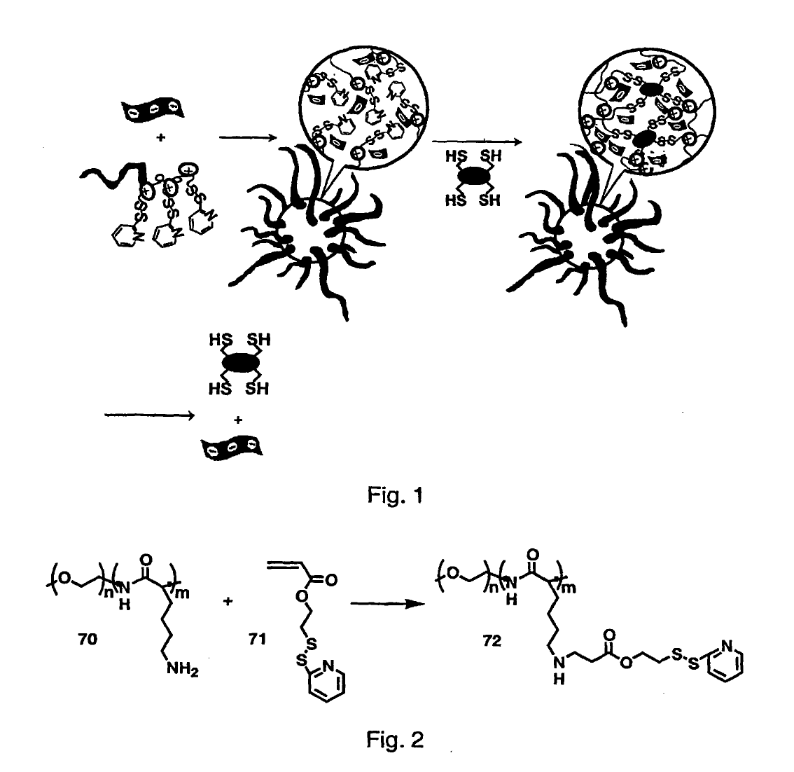

As used herein, (A)ₙ-block-(B)ₘ refers to accepted nomenclature for indicating a block copolymer where the molecules of A are found in some number n, to form a block, which is followed by molecules of B in some number, m, forming a separate block, which together form a copolymer macromolecule. In an embodiment of the present invention, the block copolymer comprises (PEG)ₙ-block-(PLL)ₘ to which dithiopyridal has been covalently attached through the amino side chains of the PLL as shown in the example presented in FIG. 1. PEG is polyethylene glycol and PLL is poly L lysine. This block copolymer is mixed with ISS DNA and micelle formation is allowed to occur. The micelle thus formed is further mixed with a peptide that is capable of being bound by APCs, such as comprising an antigenic sequence, and further comprises two cysteines residues at the amino terminus and two cysteines residues at the carboxy terminus. Under appropriate reaction conditions, each of the cysteines residues on a given peptide may react with separate dithiopyridal moiety side chains to form multiple disulfide bonds, which cross-link the copolymers, leading to micelles with cross-linked copolymers. The peptide cross-linked micelle of FIG. 1 can be used in methods of vaccination wherein a composition comprising the antigenic peptide cross-linked micelle is administered to an animal or a human and functions as an immunogenic agent. In producing an immune response, it is currently believed that the following known biological activities would occur. APCs would internalize the peptide cross-linked micelle by phagocytosis. Upon internalization, the disulfide bonds forming the cross-links are reduced by the high glutathione concentrations found within the endosomes of the APCs. The reduction of the disulfide bonds releases both the antigenic peptide and the ISS DNA to generate a specific immune response.

The peptide component of the peptide cross-linked micelle may comprise a peptide sequence suitable as a peptide antigen. The incorporation into the peptide cross-linked micelle of a specific peptide antigen would allow the targeted delivery of antigens suitable for vaccines. The sequence of such peptides could be designed by methods readily known to one skilled in the art, and the specific sequence of the peptide could be specific for the vaccine of interest. The peptide component of the peptide cross-linked micelle may also comprise a peptide sequence wherein the sequence is a substrate for a specific enzymatic reaction or a target of a specific bioactivity as discussed for reactive regions.

An aspect of the present invention comprises a synthesis of a block copolymer wherein one block is comprises a shell-forming block and a second block comprises a core-forming block. The general form of the block copolymer is (A)ₙ-block-(B)ₘ. In one aspect of the present invention, A comprises the shell-forming block and B comprises the core-forming block. In another aspect, A comprises the core-forming block and B comprises the shell-forming block. In a further aspect of the present invention, the block copolymer, (A)ₙ-block-(B)ₘ, may have an additional block polymer grafted onto the backbone, in combinations such as Xᵣ-block-(A)ₙ-block-(B)ₘ, (A)ₙ-block-(B)ₘ-block-(X)ᵣ, (A)ₙ-block-Xᵣ-block-(B)ₘ, or other combinations that are readily apparent. In yet another aspect of the present invention, the block copolymer, (A)ₙ-block-(B)ₘ, may comprise monomer units in either block A or B wherein the monomer units comprise structures with modifiable side-chains pendant to the backbone of A or B, the pendant reactive side chains. Pendant reactive side chains may be found in any block of the copolymer.

In another aspect, the shell-forming block comprises (—O—CR₁R₂—CR₃R₄—)ₙ, wherein R₁, R₂, R₃ and R₄ may be independently varied and comprise H, alkyl chains up to six carbons, polar functional groups such as hydroxyl, amines, substituted amines, sulfhydryl, carboxylic acid, amides, substituted amides, and halogens. In one embodiment, the shell-forming block is polyethylene oxide, (—O—CH₂—CH₂—)ₙ. In a further aspect, the shell-forming block comprises polyalkyl oxide, (—O—(CH₂)ₓ—)ₙ, wherein x may be 1 to 6. In yet another aspect, the shell-forming block may comprise a substituted polyalkyl oxide, (—O—(CR₁R₂)ₓ—)ₙ, wherein x may be 1 to 6, and R₁ and R₂ may be independently varied and may comprise H, alkyl chains up to six carbons, polar functional groups such as hydroxyl, amines, substituted amines, sulfhydryl, carboxylic acid, amides, substituted amides, halogens and alkyl chains up to six carbons substituted with one or more of the foregoing.

In one aspect, the core-forming block comprises a polymer of L- or D-amino acids. In a further aspect, the polymer is a homopolymer of a single amino acid. In yet another aspect, the polymer is heteropolymer of two or more amino acids. In one embodiment, the polymer is poly-L-lysine. In another embodiment, the polymer is poly-L-arginine. In a further embodiment, the polymer is a copolymer of L-lysine and L-arginine.

In another aspect, the core-forming block comprises a polyester polymer. In another aspect, the polyester is a homopolymer. In a further aspect, the polyester is copolymer of two or more distinct ester monomer units. In one embodiment, the polymer is poly(lactic acid). In another embodiment, the polymer is poly(glycolic acid). In yet another embodiment, the polymer is poly(caprolactone). In a further embodiment, the polymer is a copolymer of lactic acid and glycolic acid.

In another aspect, the core-forming polymer comprises (—NH—CR₁H—CO—)ₙ wherein R1 may comprise H, alkyl chains up to six carbons, polar functional groups such as hydroxyl, amines, substituted amines, sulfhydryl, carboxylic acid, amides, substituted amides, halogens and alkyl chains up to six carbons substituted with one or more of the foregoing. In one embodiment, R1 comprises —(CH₂)ₙNH₂ wherein x may be 1 to 12. In another embodiment, R1 comprises —(CH₂)ₓOH wherein x may be 1 to 12.

An aspect of the present invention comprises modification of the side-chains of the core-forming block by reaction of the one or more of side chains with a cross-linking moiety to form pendant reactive side chains. In one embodiment, the side-chains of the core-forming block terminate in primary amino groups which may be reacted with acrylate containing compounds, wherein the acrylate compound terminates in a suitable reactive species which is capable of reacting with a peptide containing suitable side chains. In one embodiment as exemplified in FIG. 2, the lysine side chains of PLL are allowed to react with hydroxyl-ethyl thiopyridal acrylate. The acrylate compound of the example terminates in a dithiopyridal moiety which is capable of forming covalent bonds with free sulfhydryl groups, such as those found in the side chains of the amino acid cysteine.

In another aspect of the present invention, the peptide cross-linked micelle comprises a block copolymer of the present invention, a peptide, and a nucleic acid-detectable composition complex. In one embodiment, the peptide sequence is a substrate for a targeted bioactivity response. In another embodiment, the nucleic acid-detectable composition complex comprises interaction of the detectable composition with nucleic acid through electrostatic interactions, and further electrostatic interactions between the nucleic acid and charged side chain groups of the block copolymer. In another embodiment, the nucleic acid of the composition is modified to contain a chelating group at either the 5′ or 3′ terminus of the nucleic acid sequence, wherein the chelating group complexes with a detectable construct. The detectable composition may be any of the detectable compositions taught in the present invention. The nucleic acid may include a DNA or RNA oligomer, a plasmid which is linear, circular, or supercoiled, or macromolecular RNA.

The compositions of the present invention comprise compounds or molecules which also include pharmaceutically acceptable derivatives thereof. A “pharmaceutically acceptable derivative” means any pharmaceutically acceptable salt, ester, salt of an ester, or other derivative of a compound of this invention. Derivatives of the compounds or molecules of the present invention, for example, the chelator molecules, include those that increase the bioavailability of the compositions of this invention when such compositions are administered to a human or animal (e.g., by allowing an orally administered compound to be more readily absorbed into the blood) or which enhance delivery of the composition to a biological compartment, for example, the brain or lymphatic system.

Pharmaceutically acceptable salts of the compositions of this invention include those derived from pharmaceutically acceptable inorganic and organic acids and bases. Examples of suitable acids include hydrochloric, hydrobromic, sulfuric, nitric, perchloric, fumaric, maleic, phosphoric, glycollic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acids. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N—(C₁₋₄ alkyl)⁴ salts.

The compositions of this invention may contain compounds or molecules having one or more asymmetric carbon atoms and thus occur as racemates and racemic mixtures, single enantiomers, diastereomeric mixtures and individual diastereomers. All such isomeric forms of these compounds are expressly included in the present invention. Each stereogenic carbon may be of the R or S configuration. Although specific compounds are exemplified in this application may be depicted in a particular stereochemical configuration, compounds having either the opposite stereochemistry at any given chiral center or mixtures thereof are also envisioned.

It should be understood that the compositions, such as detectable constructs, of this invention may be modified by appending appropriate chemical groups to enhance selective biological properties. Such modifications are known in the art and include those which increase biological penetration into a given biological compartment (e.g., blood, lymphatic system, central nervous system), increase oral availability, increase solubility to allow administration by injection, alter metabolism and alter rate of excretion.

It should also be understood that the compounds of this invention may adopt a variety of conformational and ionic forms in solution, in pharmaceutical compositions and in vivo. Although the depictions herein of specific compounds of this invention are of particular conformations and ionic forms, other conformations and ionic forms of those compounds are envisioned and embraced by those depictions.

The present invention comprises detectable compositions that can be prepared by combining the detectable constructs of the present invention or pharmaceutically acceptable forms thereof, together with any pharmaceutically acceptable carrier, adjuvant or vehicle. The term “pharmaceutically acceptable carrier, adjuvant or vehicle” refers to a carrier or adjuvant that may be administered to a patient, together with a detectable construct of this invention, and which does not destroy the activity thereof and is nontoxic when administered in doses sufficient to deliver an effective amount of the agent. Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, TRIS (tris(hydroxymethyl)amino-methane), partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Detectable compositions may be in the form of a sterile injectable preparation, for example a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The detectable compositions of the present invention may be administered orally, parenterally, by inhalation spray, topically, rectally, nasally, buccally, vaginally or via an implanted reservoir in dosage formulations containing conventional non-toxic pharmaceutically-acceptable carriers, adjuvants and vehicles. The term “parenteral” as used herein includes subcutaneous, intravenous, intramuscular, intra-articular, intra-synovial, intrasternal, intrathecal, intrahepatic, intralesional and intracranial injection or infusion techniques. The detectable compositions of the present invention can also be used for detection methods in ex vivo and in vitro applications wherein the above administration routes are not applicable, but similar applications are intended. For example, hydrogel detectable constructs can be used to image cellular activity in tissue culture applications.

Methods for providing imaging compositions are known in the art, and comprise providing an effective amount of the detectable composition to a human, animal or in vitro living organisms such that the targeted bioactivity is detected by the imaging technique used. The detectable compositions of the present invention can be used in methods discussed herein, including, but not limited to, imaging contrast or enhancement agents, diagnose disease states of the organs or cells of living organisms, real-time detection and differentiation of myocardial infraction versus ischemia, in vivo, i.e. whole organism, investigation of antigens and immunocytochemistry for the location of tumors, identification and localization of toxin and drug binding sites, rapid screens of the physiological response to drug therapy. Generally, sterile aqueous solutions of the compositions of the present invention are administered to a human or animal in a variety of ways, including orally, intrathecally and especially intraveneously in concentrations of 0.003 to 1.0 molar, with dosages from 0.03, 0.05, 0.1, 0.2, and 0.3 millimoles per kilogram of body weight being preferred. Dosages may depend on the structures to be imaged. Suitable dosage levels for imaging agents are outlined in U.S. Pat. Nos. 4,885,363 and 5,358,704.

Specific methods of use may comprise modifications of the compositions. For example, to increase the blood clearance times (or half-life) of the detectable agents of the carbohydrate polymers may be added to the chelator molecule (see U.S. Pat. No. 5,155,215). Additionally, other modifications may aid in crossing the blood-brain barrier. As is known in the art, a DOTA derivative which has one of the carboxylic acids replaced by an alcohol to form a neutral DOTA derivative has been shown to cross the blood-brain barrier. Thus, for example, detectable constructs are used that cross the blood-brain barrier with reaction regions that are specific for a variety of neurological disorders, including Alzeheimer's disease. Then, for example, MRI imaging could be used to correctly diagnosis Alzeheimer's disease, and provide a physiological basis to distinguish Alzeheimer's disease from depression, or other treatable clinical symptoms, or to determine tumor location, or other neurological disorders.

It must be noted that, as used in this specification and the appended claims, the singular forms “a”, “an”, and “the” include plural referents unless the context clearly dictates otherwise.

All patents, patent applications and references included herein are specifically incorporated by reference in their entireties.

It should be understood, of course, that the foregoing relates only to exemplary embodiments of the present invention and that numerous modifications or alterations may be made therein without departing from the spirit and the scope of the invention as set forth in this disclosure.

The present invention is further illustrated by way of the examples contained herein, which are provided for clarity of understanding. The exemplary embodiments should not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

### Example 1 Synthesis of Detectable Constructs

### Materials

Aidrithiol, cysteamine, 2-iminothiolane hydrochloride (Traut's reagent), and diethylenetriaminepentaacetic dianhydride (DTPA-DA) were used as received from Sigma-Aldrich. Polyethylene glycol bisamine (3,400) was used as received from Nektar, p-SCN-benzyl-diethylenetriaminepentaacetic acid (DTPA-SCN) was used as received from Macrocyclice, and 5,5′-dithio-bis-2-nitrobenzoic acid (Ellman's reagent) was used as received from Calbiochem. All columns were purchased from Amersham Biosciences: the PD-10 columns were Sephadex™ G-25M and the ion exchange columns were HiTrap™ Q FF (strong anion exchanger). Dialysis materials were purchased from Pierce with a 3,500 molecular weight cut off.

### Synthesis of Gadolinium-PEG-S—S-Dysprosium (19)

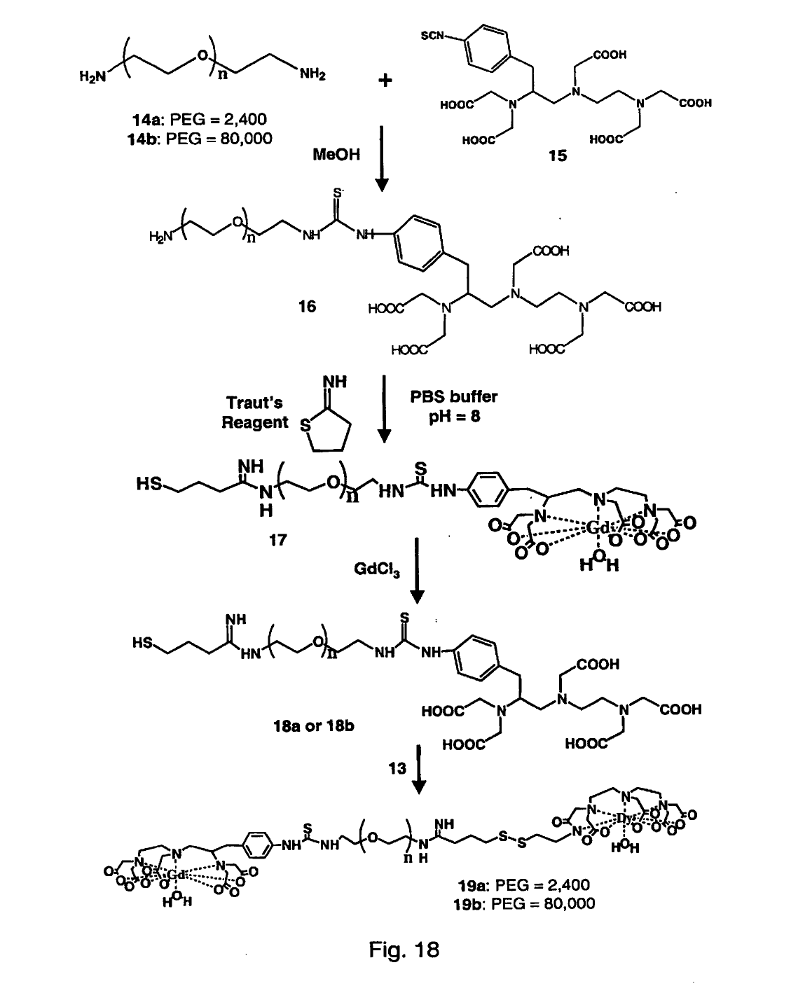

These experiments showed cleavage of the T₂/T₁ quenching detectable constructs generated a large enough change in R₂ to cause an increase in tissue signal intensity. The compounds 19a and 18a were synthesized and their R₁ and R₂ were measured. 19a represents an intact T₂/T₁ quenching detectable construct and 18a represents a detectable construct remainder of polymeric gadolinium generated after cleavage of 19a. By comparing the R₂ and R₁ of 19a and 18a, an estimate of the change in R₂ of the T₂/T₁ quenching detectable constructs after enzymatic cleavage can be obtained. The compound 19a was synthesized according to the scheme shown in FIG. 18. The synthetic details are described below.

### Synthesis of DTPA-Thiopyridine (12)

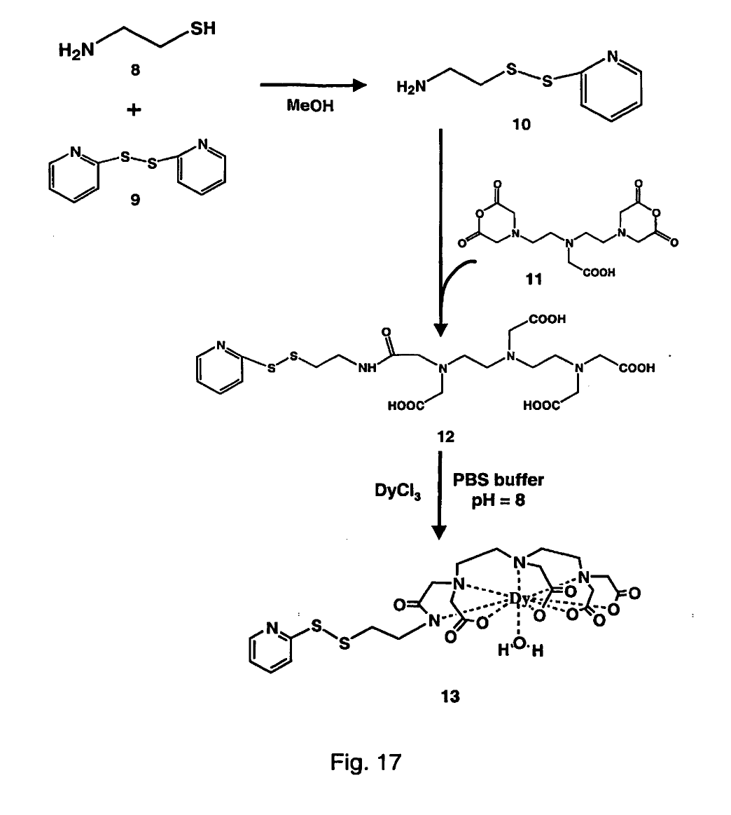

A solution of aidrithiol 4.41 g. (20.0 mmol) was prepared in 20 mL methanol and 0.8 mL acetic acid (see FIG. 17). Another solution of 1.14 g (10.0 mmol) of cysteamine in 10 mL of methanol was prepared and then added dropwise over 30 minutes to the aldrithiol solution. The reaction mixture was allowed to stir for 4 hours after which the product was separated by silica gel chromatography. The column was eluted with 100% ethyl acetate to remove impurities and then eluted with methanol/triethylamine (80:20 ratio) to yield aminoethyl-thiopyridine in 60% yield (10). ¹H NMR (deuterated DMSO) peaks were ζ 1.83 (2H, s), ζ 2.717 (2H, d, J=6.4 Hz), ζ 2.784 (2H, d, J=6.0 Hz), ζ 7.263 (1H, t, J=4.8 Hz), ζ 7.593 (1H, d, J=8 Hz), ζ 7.793 (1H, t, J=4.8 Hz), ζ 8.461 (1H, d, J=4 Hz).

The compound 10 (1.0 mmol) was then reacted with 2.0 mmol of DTPA-dianhydride (11) in 0.1M sodium bicarbonate buffer at pH 8.5. The reaction was allowed to proceed for 3 hours at room temperature and was purified by ion exchange chromatography. The reaction solution was loaded onto the ion exchange column at pH 8.5 (0.1M sodium bicarbonate buffer) and eluted with acetate (0.01M, 100 mM NaCl) at pH 2.9. The presence of the product was monitored by UV-Vis spectrometry and fractions were collected to obtain product 12 in 35% yield. Molecular weight from electrospray mass spectrometry: 562.0, formula weight: C₂₁H₃₁N₅O₉S₂=562.0.

### Synthesis of Thiol-PEG-DTPA-Gd (18A)

As shown in FIG. 18, Polyethylene glycol bisamine (14a) (0.1 g 0.03 mmol) was added to 2 mL of DMSO dried over 4 molecular sieves. DTPA-isothiocyanate (15) (0.01 0.015 mmol) was then added to 14a in a 2:1 excess. The reaction was allowed to mix for 24 hours and was run through a PD-10 column, with deionized water as the eluent, to remove unreacted 15. The high molecular weight fractions of the PD-10 column were collected and then purified by ion exchange chromatography to remove unreacted 14a. The column (High Trap, anion exchange-Pharmacia) was equilibrated with 0.01M Tris buffer at pH 9 and the crude product was loaded, and then washed with 5 column volumes of 0.01M Tris buffer at pH 9. The product was eluted with 0.01M acetate buffer (100 mM NaCl) at pH 3, and lyophilized overnight generating 16. The compound 16 (25 mg, 0.00625 mmol) was then reacted with Traut's reagent (4 mg/ml) in 0.5 mL pH 8 phosphate buffer solution (50 mM) for 3 hours, to generate the thiol terminated 17; total yield was 23% for both steps as determined by Elfrnan's assay. 20 mg (0.054 mmol) of GdCl₃ (Alfa Aesar) was added to the crude 17 solution and was allowed to mix for 30 minutes. The compound 18 was purified by a PD-10 column (using deionized water as the eluting agent).

### Synthesis of Gd-PEG-S—S-Dy (19A)

The compound 19a was synthesized by reacting the thiol of 18 with the thiopyridal group of 13. The compound 18 was dissolved in a sodium bicarbonate solution (pH 8, 0.1M) at a 12.4 mg/ml (1.0 mM) concentration. The compound 12 was dissolved in water at 5.9 mg/ml (10 mM) and then reacted with excess DyCl₃ (Alfa Aesar) to generate 13 After 30 minutes the entire solutions of 13 and 18 were mixed together and allowed to react overnight, and then purified by a PD-10 column to yield 19a. The reaction was monitored by measuring the release of the thiopyridone by U.V. absorbance at 342 nm. The yield was 95% based on UV.

### Example 2R₂ and R₁ Measurements of 18A and 19A

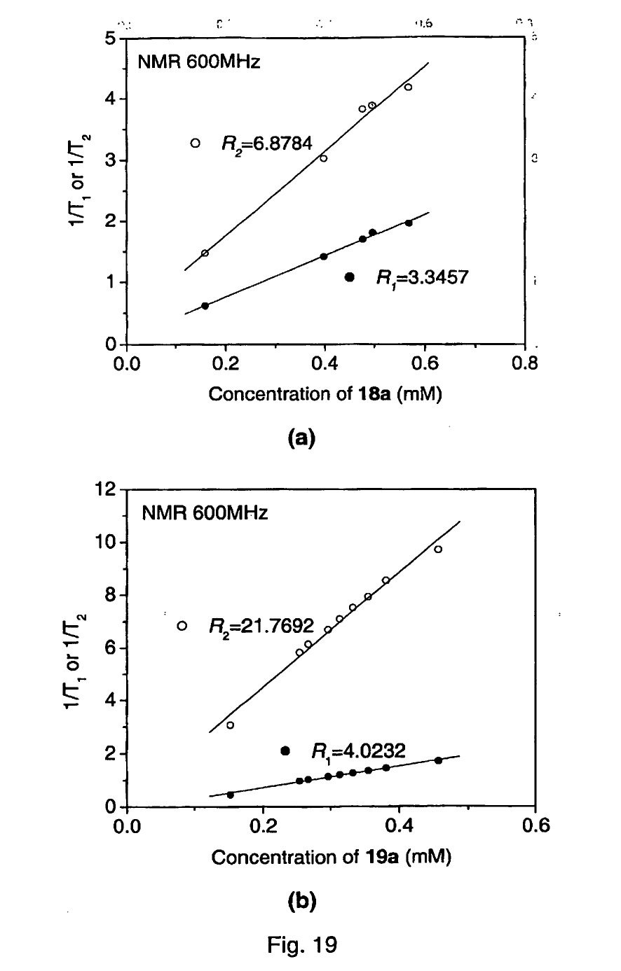

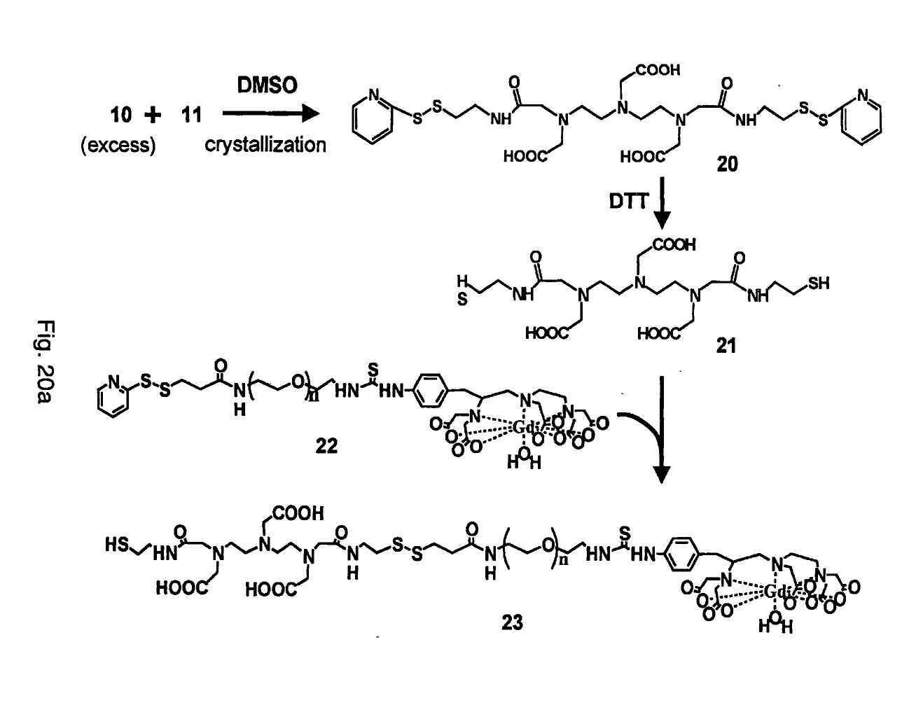

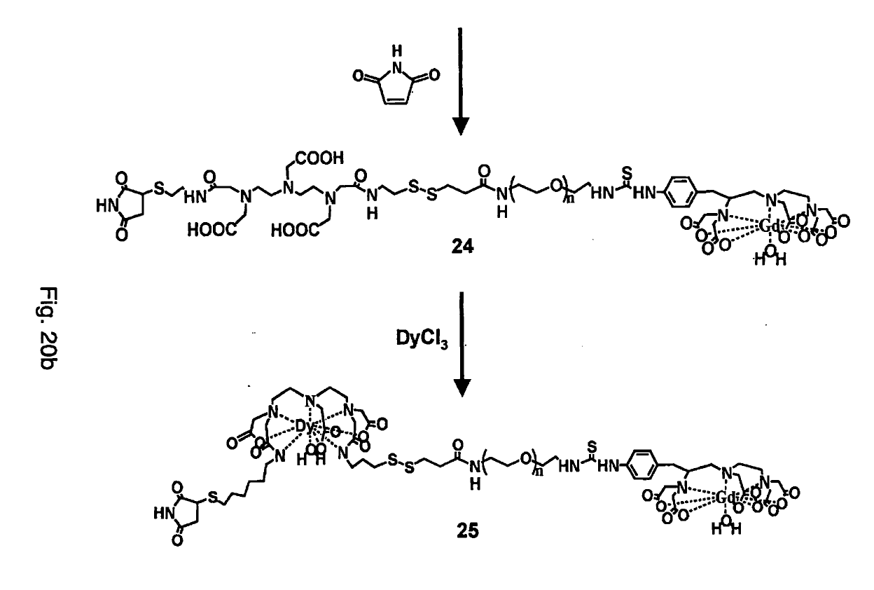

The R₂ and R₁ of 19a and 18a were calculated to show cleavage of the T₂/T₁ quenching detectable construct 19a generated a large enough change in R₂ to allow for enzyme detection. The compound 19a represents an intact T₂/T₁ quenching detectable construct and the compound 18a represents a polymeric gadolinium detectable construct remainder resulting from cleavage of the reaction region. The R₁ and R₂ of 19a and 18a were measured in deuterium oxide in a Unity-600 MHz (Varian, Inc.) at 25±0.1° C. T₁ and T₂ measurements of water relaxation times were measured at various concentrations of 19a and 18a, and a plot of 1/T₁ and 1/T₂ was made against the concentration; the relaxivities R₁ and R₂ were determined by the slope of this graph using equation 1 (Caravan 1991),

$$\frac{1}{{T}_{i}} = \frac{1}{{T}_{i} ( diamangetic )} + {R}_{i} ( {C}_{i} ) where i = 1 or 2 \qquad Equation 1$$

where 1/T₁ is the relaxation rate of the water proton induced by the paramagnetic and diamagnetic species present in solution, and 1/T₁ (diamagnetic) is the relaxation rate in the absence of the paramagnetic species. R₁ and R₂ of 18a were calculated to be 3.34 mM⁻¹s⁻ and 6.8 mM⁻¹s⁻¹ and the R₁ and R₂ of 19a were 4.0 mM⁻¹s⁻¹ and 21.77 mM⁻¹s⁻¹ (see FIG. 19).

This study demonstrated that the T₂/T₁ quenching methods generated a large change in R₂ and a small change in R₁ after cleavage of a dysprosium detectable construct remainder from a quenching detectable construct, and had the sensitivity required to detect enzymes through bioactivity which cleaves the reaction region. For example, the compound 19a, which represents an intact T₂/T₁ quenching detectable construct, has an R₂ of 21.77 mM⁻¹s⁻¹ whereas the R₂ of 18a, which represents a detectable construct remainder of a T₂/T₁ quenching detectable construct, is only 6.8 mM⁻¹s⁻¹. This represented a 69% reduction in R₂ with cleavage of the T₂/T₁ quenching detectable construct. Importantly the decrease in R₁ between 19a and 18a was only 16.5%, going from 4.0 mM⁻¹s⁻¹ to 3.34 mM⁻¹s⁻¹. Thus the data demonstrated that the T₂/T₁ quenching methods and compositions generated a measurable change in signal intensity after cleavage, particularly after optimization of the TE and TR times.

### Example 3 T₂/T₁ Signal Quenching Methods

### Imaging Dithiothreitol (DTT)

In this study, a T₂/T₁ quenching detectable construct and method is tested, using a T₂/T₁ quenching detectable construct designed to image the disulfide cleaving reagent DTT. Cleavage of the T₂/T₁ quenching detectable construct gadolinium-PEG-S—S-dysprosium (19a) by DTT results in an increase in MRI signal intensity and allows the imaging of DTT through MRI. The cleaved dysprosium detectable construct remainder increases its translation diffusion coefficient and diffuses away from the gadolinium detectable construct remainder. Cleavage of the rotationally hindered T₂/T₁ quenching detectable construct, gadolinium-poly(methacrylic acid)-S—S dysprosium (33) by DTT results in increased signal intensity and the imaging of DTT through MRI. The cleaved dysprosium detectable construct remainder will have a decreased rotational correlation time and R₂.

Three T₂/T₁ quenching detectable constructs are synthesized (19b, 25 and 33), (See FIGS. 18, 20 and 21) and their effects on MRI signal intensity are investigated in the presence and absence of DTT. The compounds 19b and 25 are tested in agarose gels to determine that the dysprosium detectable construct remainder generated from DTT cleavage has a large enough increase in its translational diffusion coefficient to diffuse away from the polymeric gadolinium detectable construct remainder 18 and causes an increase in signal intensity. The compound 33 is tested in agarose hydrogels to determine that the dysprosium detectable construct remainder generated from DTT cleavage has a decrease in its rotational correlation time and R₂.

### Synthesis of Gadolinium-PEG-S—S-Dysprosium (19B)

The compound 19b is identical to the previously synthesized 19a, except that it has a PEG of molecular weight 80,000 instead of 3,400. 19b is synthesized following the synthetic procedure used for 19a. The starting material H₂N-PEG-N₂H (80,000) (14b) for 19b is synthesized according to procedure described by Greenwald et al (1996) and Bertozzi et al (1991). See FIG. 18.

### Synthesis of Gadolinium-PEG-S—S-Dysprosium-Diamide (25)

The T₂/T₁ quenching detectable construct 25 is synthesized as shown in FIG. 20. The dysprosium chelate 25 has two amide bonds and had a water exchange rate τₘ that is 34 times lower than the dysprosium chelate in 19b, which has one amide bond in its dysprosium chelate. This is a derivative form of DTPA. The chelator molecule of this example is considered a modification of the detectable detectable agent, wherein the chelator molecule has substitutions. The R₂ of dysprosium chelates is inversely proportional to the water exchange rate, particularly for macromolecular dysprosium chelates at high field strengths (Vander 2002 and Caravan 2001) The dysprosium chelate detectable construct remainder in 25 has a greater R₂ than the dysprosium chelate detectable construct remainder in 19b and also generates a larger change in tissue T₂ after cleavage and diffusion away from the polymeric gadolinium detectable construct remainder. The compound 25 is synthesized according to the scheme shown in FIG. 20. The R₁ and R₂ of 25 is determined using a 600 MHz NMR machine as described in Example 2.

### Synthesis of Gadolinium-Poly(Methacrylic Acid)-PEG-S—S-Dysprosium (33)

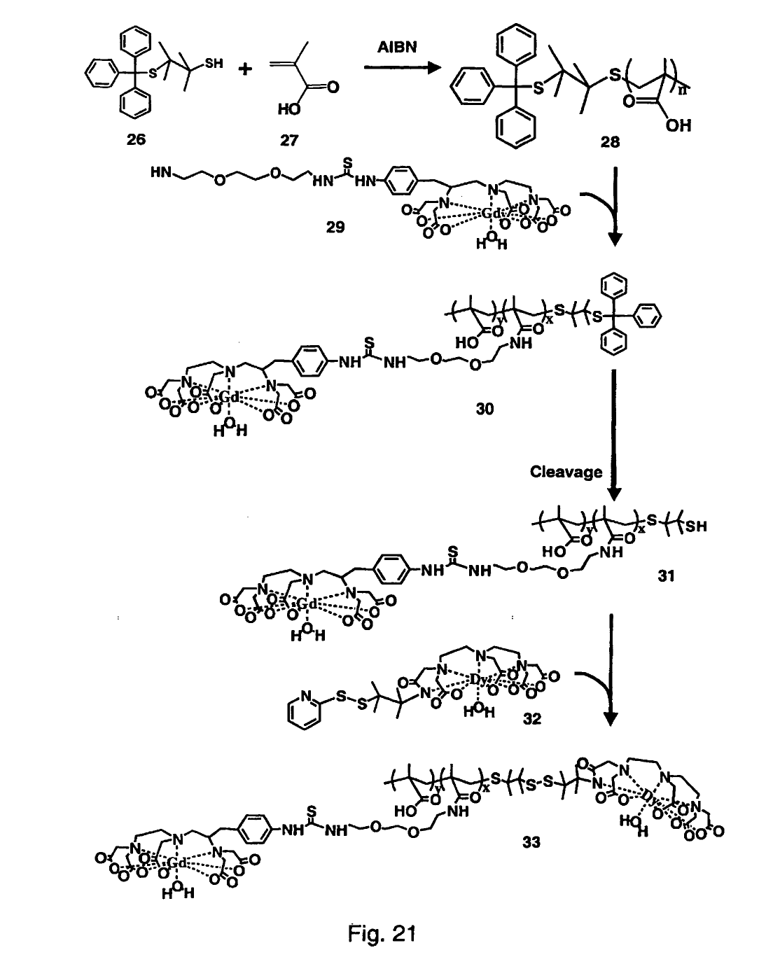

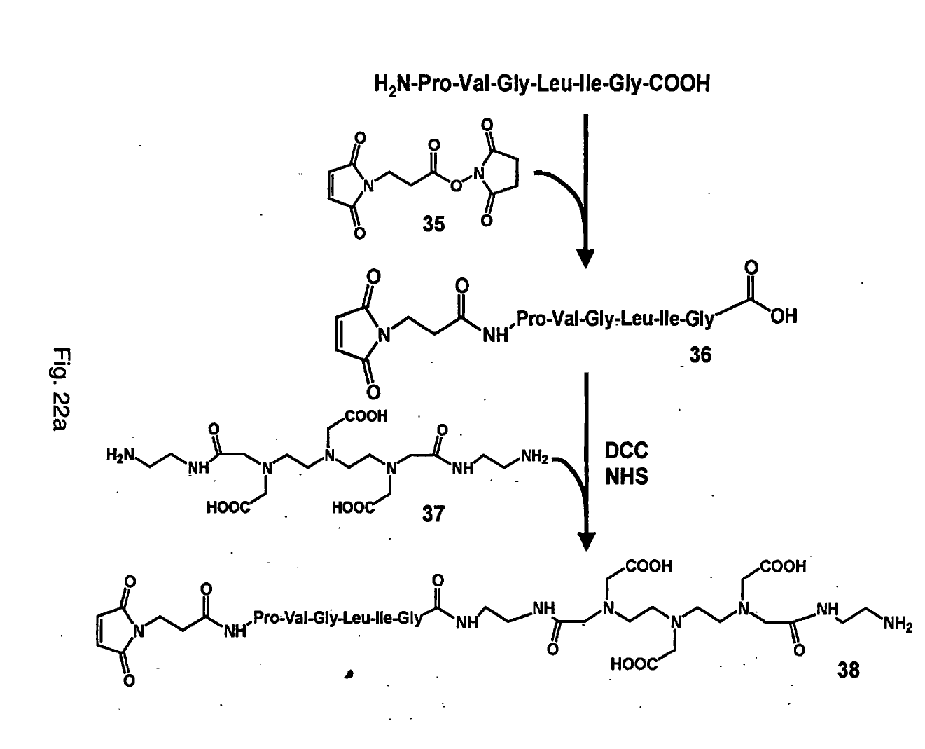

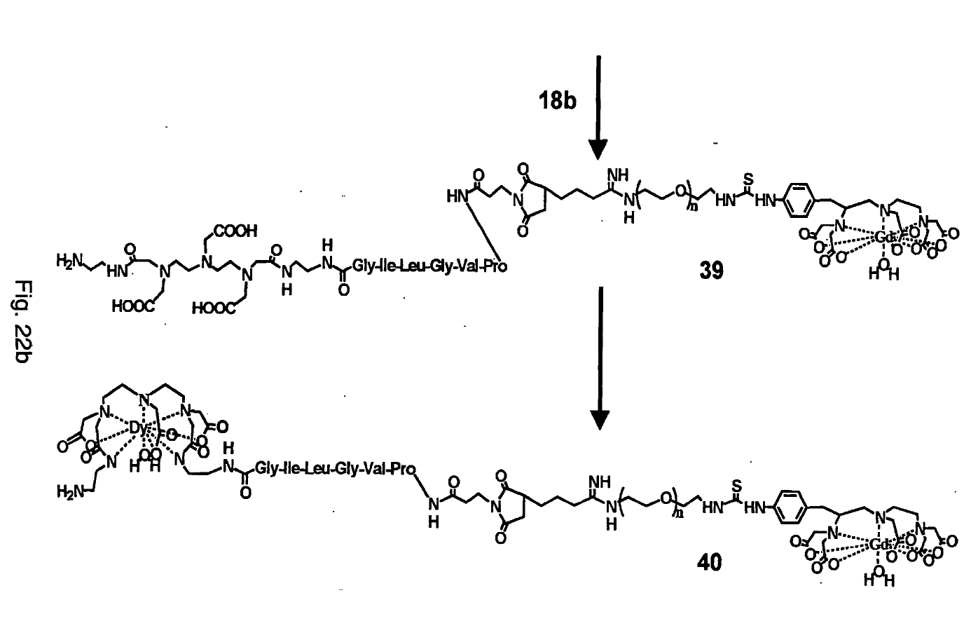

The rotationally hindered T₂/T₁ quenching detectable construct 33 is synthesized according to the scheme shown in FIG. 21. The rotationally hindered chain transfer agent 26 is polymerized with methacrylic acid to generate 28, which is purified by precipitation in ether. The gadolinium chelate 29 is coupled to the carboxyls on 28 using DCC and NHS, in DMSO, and is isolated by dialysis. The compound 30 is deprotected with acid to generate the thiol 31, which is coupled to 32. The final product 33 is isolated by dialysis and freeze dried. See FIG. 21.

### Example 4 Imaging DTT with 19B and 25 in Agarose Gels

The T₂/T₁ quenching detectable constructs 19b and 25 are loaded into 2% agarose hydrogels at a concentration of 0.5 mM. The hydrogels have a volume of 0.5 ml and are made in 10 ml scintillation vials. After the hydrogels form, the vials are filled with PBS buffer, pH 7.4, and then imaged in either a 9.4 Tesla MRI machine or a 17.8 Tesla MRI machine (University of Florida). The best TR/TE parameters are selected from calculations using the spin-echo signal equation with respect to the relaxation times and the TR/TE. The TE and TR are chosen such that the signal intensity of the hydrogels loaded with 19b and 25 is lower than or equal to pure agarose hydrogels. These calculations are based on the R₁ and R₂ of 19b and 25. DTT is added to the scintillation vials to generate a 50 mM concentration of DTT in the vials. The control hydrogels for this experiment consist of hydrogels that contain 50 mM DTT, without 19b or 25. The signal intensity of the gels is measured every hour for 48 hours. The T₁ and the T₂ of the gels is measured every hour, the T₁ is measured using an inversion recovery sequence, for which the sequence parameters will be selected based on the values of R₁ and R₂ of 19b and 25. The T₂ will be measured using a Carr-Purcell-Meiboom-Gill (CPMG) sequence with TEs chosen from the values of R₂ for 19b and 25 (Haacke 1999).

The change in signal intensity, T₂, and T₁ of the agarose hydrogels after the addition of DTT indicates that the cleaved dysprosium detectable construct remainder diffuses out of the gel, while the gadolinium-PEG detectable construct remainder remains inside. The addition of DTT increases the signal intensity of the gels with 19b and 25 and causes their signal intensity to be greater than the controls (agarose +DTT).

### Example 5 Imaging DTT with 33

An experimental procedure similar to that outlined in Example 4 is used to image DTT, except that the hydrogels are loaded with compound 33. The scintillation vials which contain the gels are not filled with buffer. This ensured that the cleaved dysprosium remains in the gel and that changes in T₂ and signal intensity are due to changes in the rotational correlation time of dysprosium. The DTT is directly added to hydrogels as a concentrated solution. The gels are analyzed in a manner similar to Example 4. The control for this experiment is agarose hydrogels that just contain DTT.

The change in signal intensity, T₂ and T₁ of the agarose hydrogels after the addition of DTT indicates that the dysprosium detectable construct remainder decreases its rotational correlation time after cleavage. In particular, the addition of DTT increases the signal intensity of the gels with 33 and causes their signal intensity to be greater than the controls (agarose +DTT). Problems with synthesis of 33 due to its high polydispersity may result from the free radical polymerization used to generate 28. This problem can be solved by using atom transfer radical polymerization (ATRP) to synthesize 28, using a protected methacrylic acid monomer and an (ATRP) initiator with a functional handle.

### Example 6 T₂/T₁ Quenching Detectable Constructs for the Detection of MMP-9

T₂/T₁ quenching detectable constructs that contain a reaction region with the MMP-9 substrate peptide with the sequence of SEQ ID NO: 1, Pro-Val-Gly-Leu-Ile-Gly, are used. Cleavage of the T₂/T₁ quenching detectable construct, gadolinium-PEG-Pro-Val-Gly-Leu-Ile-Gly-dysprosium (40) by MMP-9 results in an increase in signal intensity and the imaging of MMP-9 with MRI. The cleaved dysprosium detectable construct remainder increases its translational diffusion coefficient and diffuses away from the macromolecular gadolinium detectable construct remainder. Cleavage of the rotationally hindered T₂/T₁ quenching detectable construct, gadolinium-poly(methacrylic acid)-Pro-Val-Gly-Leu-Ile-Gly-dysprosium (42), by MMP-9 results in an increased signal intensity and the imaging of MMP-9 with MRI. The cleaved dysprosium detectable construct remainder has a lower rotational correlation time and R₂ than the gadolinium-poly(methacylic acid)-Pro-Val-Gly-Leu-Ile-Gly-dysprosium quenching detectable construct, resulting in an increase in T₂ and MRI signal intensity.

T₂/T₁ quenching detectable constructs, gadolinium-PEG-Pro-Val-Gly-Leu-Ile-Gly-dysprosium (40) and gadolinium-poly(methacrylic acid)-Pro-Va-Gly-Leu-Ile-Gly-dysprosium (42) are synthesized and used to image MMP-9 in agarose hydrogels. The compounds 40 and 42 are similar to the thiol sensitive T₂/T₁ quenching detectable constructs 33 and 19 except that the reaction region is an MMP-9 substrate, Pro-Val-Gly-Leu-Ile-Gly. The peptide Pro-Val-Gly-Leu-Ile-Gly is a well characterized MMP-9 substrate and has been used for the synthesis of MMP-9 activated prodrugs; it has a k_{cat}/Kₘ of 7.09×10³ M⁻¹s⁻¹ for cleavage by MMP-9 from a dextran backbone (Chau 2004).

### Synthesis of Gadolinium-PEG-Pro-Val-Gly-Len-Ile-Gly-Dysprosium (40)

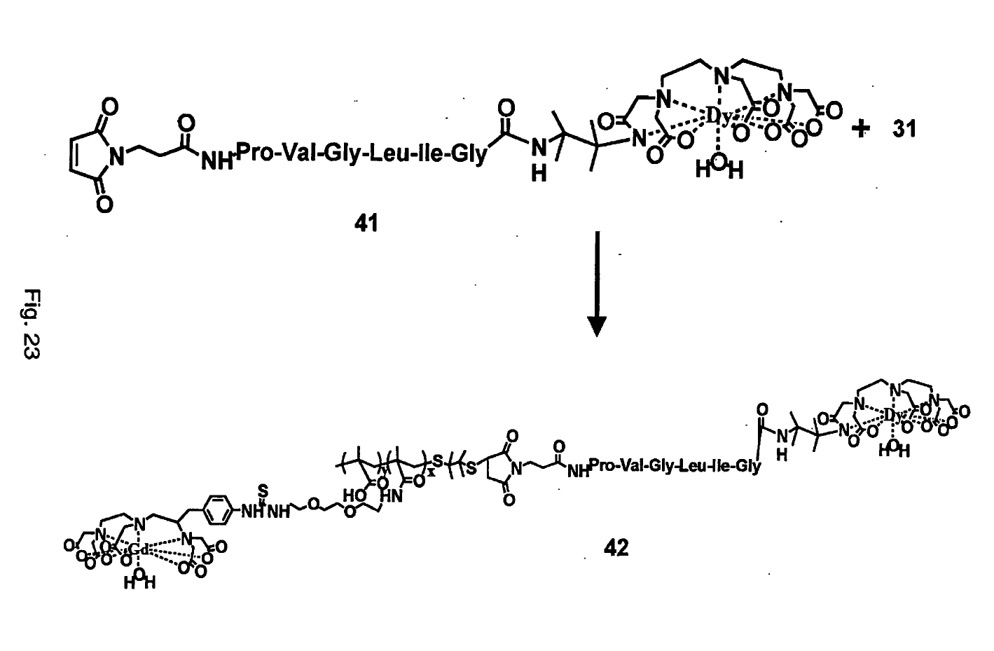

The compound 40 will be synthesized according to the scheme shown in FIG. 22. The peptide H₂N-Pro-Val-Gly-Leu-Ile-Gly-COOH (34) will be purchased from the SynPep Corporation. Its terminal amine will be reacted with NHS-ethyl-maleimide (35) (purchased from Pierce Chemicals) in dry DMF. The product (36) is purified by preparative HPLC, using a Jupiter column from Phenomenex Corporation. The carboxyl of 36 is then reacted with a 10 fold excess of DTPA-diamine (37), in DMSO, using NHS and DCC as the coupling reagents, the product (38) is purified by preparative HPLC. The maleimide group of 38 is coupled to the thiol group of 18b, in DMSO, and the product is first purified by gel filtration chromatography (PD-10 column, Pharmacia) to remove unreacted 38. An excess of 38 is used in this reaction, to ensure that 18b is quantitatively reacted; however, if there remains unreacted 18b, then HPLC is used to purify 39. The final product 40 is obtained by chelating 39 with excess dysprosium chloride, in water, and the product 40 is purified by gel filtration chromatography (PD-10 column Pharmacia) and characterized by mass spectrometry (ES). The k_{cat}/Kₘ of this substrate for MMP-9 is measured according to the procedure outlined in Chau et al (2004).

### Synthesis of Gadolinium-Poly(Methacrylic Acid)-Pro-Val-Gly-Leu-Ile-Gly-Dysprosium (42)

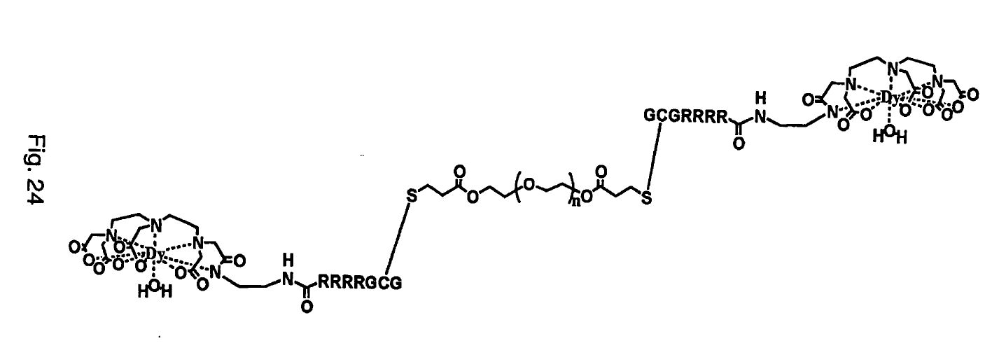

The T₂/T₁ quenching detectable constructs 42 is synthesized according to the scheme shown in FIG. 23. Compound 41 is synthesized by reacting compound 38 with dysprosium (DyCl₃) and the product is purified by HPLC. The compound 41 is reacted with compound 31 in DMSO, using triethylamine as a base. An excess of 41 is used to ensure that 31 is reacted to completion. The product 42 is purified by dialysis. The k_{cat}/Kₘ of this substrate for MMP-9 is measured according to the procedure outlined in Chau et al. (2004)

### Example 7 Imaging MMP-9 with 40 in Agarose Gels

The T₂/T₁ quenching detectable constructs 40 and MMP-9 are loaded into 2% agarose hydrogels at a concentration of 0.5 mM and 0.5 μM. The hydrogels have a volume of 0.2 ml, and are made in a 5 ml scintillation vial. After the hydrogels form, the vials are filled with PBS buffer, pH 7.4, and then imaged in either a 9.4 Tesla MRI machine or a 17.8 Tesla MRI machine (University of Florida). The control hydrogels for this experiment are hydrogels that contain 40, without MMP-9 and hydrogels composed of just water. Numerical calculations are conducted using the spin-echo signal equation with respect to the relaxation times and the TR/TE. The TE and TR is chosen such that the signal intensity of the hydrogels loaded with 40 is lower than or equal to that of pure agarose hydrogels. The signal intensity of the gels is measured every hour for 48 hours. The T₁ and the T₂ of the gels is measured every hour, with T₁ being measured using an inversion recovery sequence, for which the sequence parameters will be selected based on the values of R₁ and R₂ of 40. The T₂ is measured using a CPMG sequence with TEs chosen from the values of R₂ for 40. If the signal intensity of hydrogels composed of MMP-9 and 40 is greater than the signal intensity of the two controls, then MMP-9 cleaved 40 and the released dysprosium successfully diffused away from the polymeric gadolinium.

### Example 8 Imaging MMP-9 with 42 in Agarose Gels

An experimental procedure similar to that outlined in Example 7 is used to image MMP-9, except that the hydrogels are loaded with compound 42. The scintillation vials which contain the agarose hydrogels are not filled with buffer. This will ensure that the cleaved dysprosium remains in the agarose hydrogel and that changes in T₂ and signal intensity are due to changes in the rotational correlation time of cleaved dysprosium. The gels are analyzed in a manner similar to Example 7. The control hydrogels for this experiment are hydrogels that contain 42, without MMP-9, and hydrogels composed of just water. If the signal intensity of hydrogels composed of MMP-9 and 42 is greater than the signal intensity of the two controls, then MMP-9 cleaved 42 and the released dysprosium increased the MRI signal intensity due to a decrease in its rotational correlation time.

Alternative T2/T₁ quenching detectable constructs, which have a lower molecular weight PEG, instead of the 80,000 PEG used in 40, can be used as can a neutral rotationally hindered polymer backbone instead of PMMA in 42. Additionally, different peptide substrates can be used to enhance cleavage by MMP-9 (Kridel 2001).

### Example 9A Dy Only Detectable Construct

A trypsin T2/T1 quenching detectable construct that contains one or more dysprosium detectable agents and does not contain the T1 contrast agent gadolinium is shown in FIG. 24. This trypsin, Dy only, detectable construct is chosen for investigation because it will show the direct effects enzyme cleavage has on the R2 of the dysprosium chelate, before and after cleavage of the detectable construct. Furthermore, this compound can be used in methods where a detectable composition comprising subcompositions is administered. For example, the subcomposition comprises a detectable construct comprising a cleavable PEG-peptide-dysprosium and a second subcomposition comprises a PEG-gadolinium detectable construct, and the two subcompositions are administered simultaneously or concurrently, in place of using a detectable composition comprising the more complex gadolinium-PEG-peptide-dysprosium quenching detectable construct as described in FIG. 14.

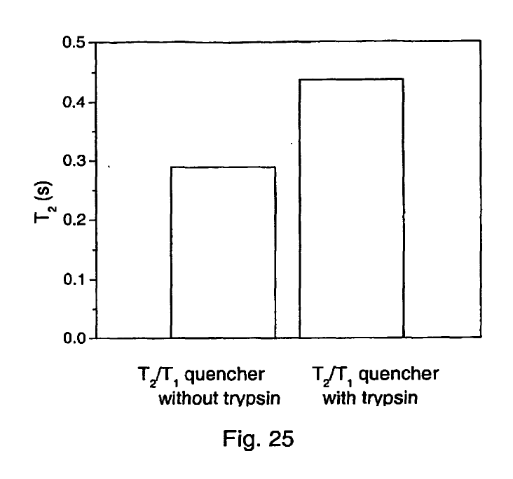

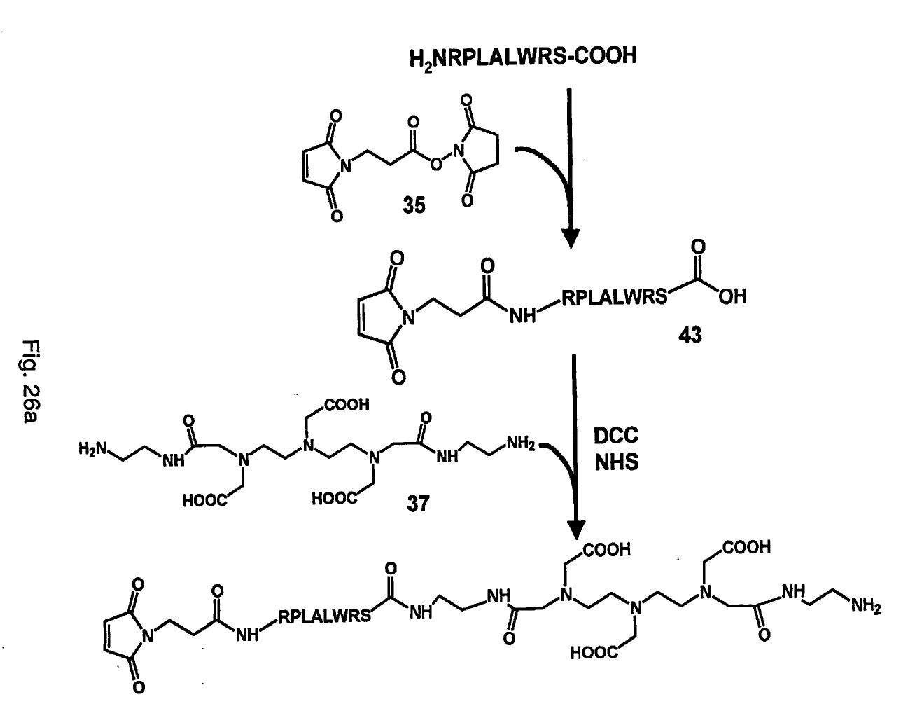

FIG. 25 demonstrates that the detectable construct can be expected to detect the presence of trypsin activity by MRI. The striped bar represents the water T2 in the presence of a 16 mg/ml concentration of the Dy only detectable construct, and the black bar represents the water T2 after addition of the trypsin to the Dy only detectable construct solution. As shown in the figure, the water T2 increases by approximately 52% after the addition of trypsin, which reflects a 52% decrease in the R2 of the Dy only detectable construct after enzyme cleavage, which can be detected by MRI. In FIG. 25, the Dy only detectable construct, without and with enzyme, are measured in a 400 MHz Oxford NMR in D2O. The concentration of the Dy only detectable construct is 16 mg/ml. Trypsin concentration is 0.025 mg/ml. After adding trypsin, T2 increases by 52.3%, which is detectable by MRI.

### Example 10 In Vivo Imaging of MMP-7 Activity in Mouse Tumors

MMP-7 activity is imaged in athymic nude mice which have been given MMP-7 expressing xenograft tumors, using the T₂/T₁ quenching detectable construct, gadolinium-PEG-RPLALWRSC-dysprosium (46, FIG. 26). This compound contains a reaction region that is the MMP-7 cleavage site, RPLALWRSC, (SEQ ID NO: 2). The expectation is that cleavage of 46 by MMP-7 will result in an increase in MRI signal intensity and the imaging of MMP-7 activity. See FIG. 26 for the synthesis of 46.

The tumors are established by subcutaneous injection of 1×10⁶ MMP-7 expressing SW480mat cells or control SW480neo cells, which do not express MMP-7, in the flanks of athymic mice. The resulting tumors are allowed to grow up to a size of 1-2 cm (4-6 weeks). During this period, the mice are imaged at various times points ranging from 3 days to 6 weeks after the injection of the tumor cells, according to the following procedure: after proper anesthetization, a catheter is inserted into the tail vein of the mice, a composition comprising the detectable construct 46 is injected through the catheter and the mice are imaged every hour for 24 hours in a 9.4 Tesla MRI machine (Emory University), measuring the MRI signal intensity, tissue T₁ and tissue T₂.

The signal intensity and T₂ of the MMP-7 expressing tumors is compared against the control tumors. If the signal intensity of the MMP-7 expressing tumors is greater than the controls, then MMP-7 successfully cleaved 46, resulting in an increase in MRJ signal intensity and the imaging of MMP-7. Furthermore, data is obtained relating to the sensitivity of the method to image early tumor activity, as well as ratiometric information relating to tumor growth.

### Example 11 Synthesis of PEG-PLL(Thiopyridyl)

### Materials

ε-(Benzyloxycarbonyl)-L-Lysine was purchased from Novabiochem, α-methoxy-ω-amino-poly(ethylene glycol) (PEG, MW=5,000) was purchased from NEKTAR Transforming Therapeutics. Immunostimulatory DNA with the sequence of SEQ ID NO:3, TCCATGACGTTCCTGACGTT, was purchased from Intergrated DNA Technologies. Agarose was purchased from Bio-Rad, methanesulfonic acid was purchased from Acros, anisole and triphosgene were purchased from TCI America, 5,5′-dithio-bis(2-nitrobenzoic acid) was purchased from Calbiochem (Ellman's Reagent), 2,2-dithiodipyridine was purchased from Fluka, sodium hydroxide, potassium hydroxide, sodium phosphate monobasic and acetonitrile were purchased from Fisher Scientific. L-Glutathione (GSH), dithiothreitol (DTT), poly(vinyl sulfate potassium salt) (PVS, MW=17,000), acetic acid, trifluoroacetic acid, 2-mercaptoethanol, dichloromethlylene, tetrahydrofuran (THF) and N,N-dimethylformamide (DMF) were purchased from Sigma-Aldrich. THF and DMF were doubly distilled before use. The peptide with the sequence of SEQ ID NO:4, Cys-Gly-Cys-Arg-Ile-Gln-Arg-Gly-Pro-Gly-R-Ala-Phe-Val-Thr-Ile-Gly-Lys-Cys-Gly-Cys-Gly, was synthesized at the Petit Institute for Bioengineering and Bioscience's core facility on a 433A Peptide Synthesizer, Applied Biosystems, and was purified by HPLC using a reversed phase column (Discovery HS C18, made by Supelco) on Hewlett Packard Series 1090 Liquid Chromotograph instrument.

### Synthesis of PEG-PLL(Thiopyridyl)

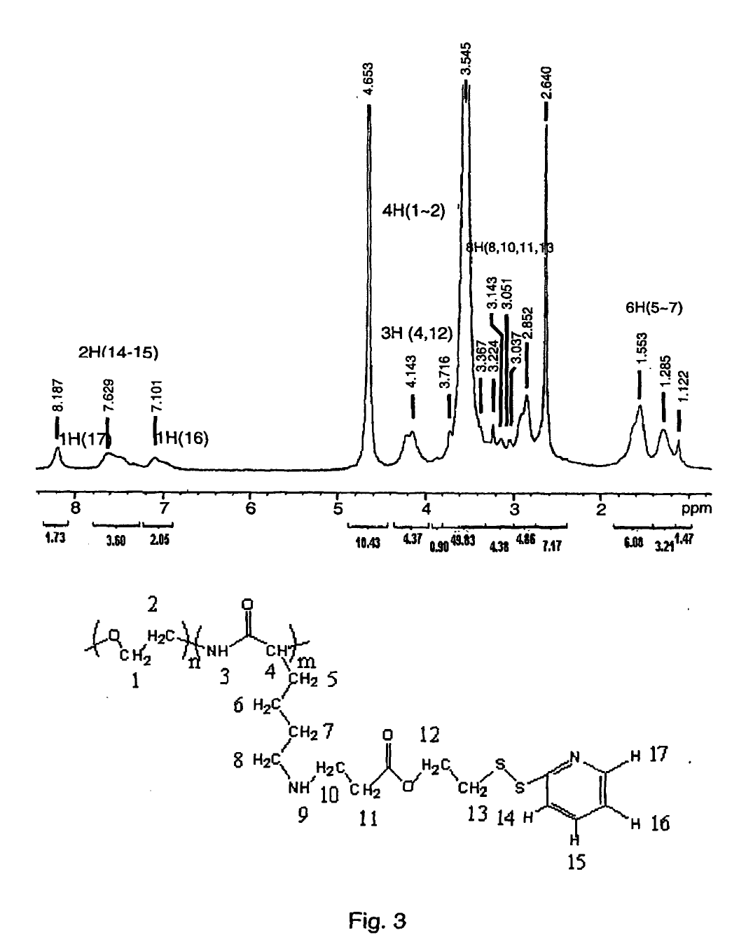

The reaction scheme is shown in FIG. 2. In general, about 162 mg (approximately 415 μmole) of PEG-PLL (Compound III in FIG. 2) was synthesized according to Harada et al. using α-methoxy-ω-amino-poly(ethylene glycol) (PEG, MW=5,000) and poly-1-lysine (MW=5,000). PEG-PLL was dissolved in 1 ml of DMF contained in a 5 ml round bottom flask and fitted with a stir bar. Overnight stirring at room temperature was required to completely dissolve the PEG-PLL polymer. To the dissolved polymer, PEG-PLL, were added about 100 mg (approximately 415 mmole) of 2-(pyridin-2-yldisulfanyl)ethyl acrylate (Compound IV in FIG. 2) and about 60 μl of triethylamine, and the reaction was allowed to proceed for 24 hours at room temperature with stirring. The product was isolated by precipitating the reaction solution into 15 ml of ice cold diethyl ether. The yield was about 88%. The product was analyzed by ¹H NMR in D₂O and a representative spectra is shown in FIG. 3. The percentage of alkylation of amine groups was determined by comparing the peak intensity ratio of pyridine protons (—NC₅H₄: δ=7.101 ppm, 7.629 ppm, 8.187 ppm) versus α, β, γ-methylene protons of PLL (—CH₂CH₂CH₂: δ=1.122 ppm, 1.285 ppm, 1.553 ppm). The data indicated that about 100% of the amines had been alkylated by reaction 2-(pyridin-2-yldisulfanyl)ethyl acrylate, yielding PEG-PLL(thiopyridyl) (Compound I in FIG. 2).

### Example 12 Dynamic Light Scattering Analysis of Peptide Crosslinked Micelles

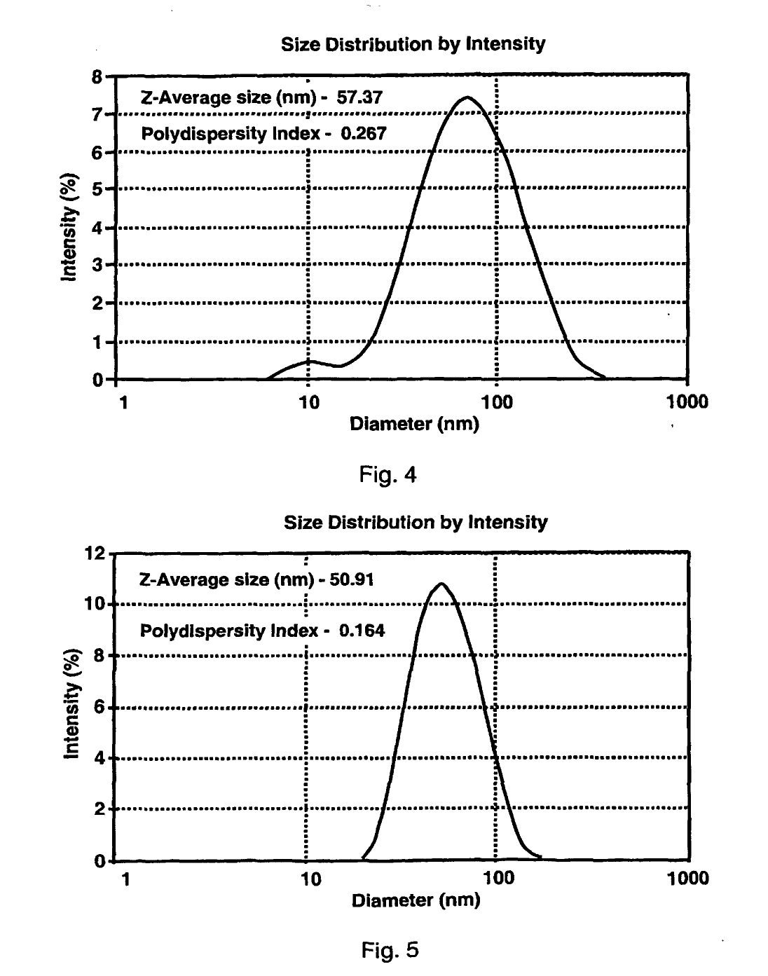

A 1 ml solution containing 50 mM phosphate-buffered saline, pH 7.4, 0.06 mg of PEG-PLL(thiopyridyl) as prepared in Example 11, and 20 μg of ISS DNA was prepared and filtered through a 200 nm syringe filter. The solution was incubated in a 1.5 ml microcentrifuge tube at room temperature for about 2 hours without agitation. The ISS DNA used in this example is as given in Example 11. The micelles formed from self-assembly of PEG-PLL(thiopyridyl) were then analyzed by dynamic light scattering (Zetasizer Nano ZS, Malvern Instruments) using the Cumulant method. The size and the size distribution of the non-crosslinked micelles are shown in FIG. 4. The micelles in the solution were then crosslinked by adding 0.12 mg of peptide and the reaction proceeded for about 3 hours at room temperature without agitation. The micelles following crosslinking with peptide were analyzed by dynamic light scattering. The size and the size distribution of the peptide crosslinked micelles (“PCM”) are shown in FIG. 5. The peptide used in these experiments with sequence of SEQ ID NO:4, CGCRIQRGPGRAFVTIGKCGCG. The portion of the peptide sequence corresponding residues 4 to 18 of SEQ ID NO:4, RIQRGPGRAFVTIGK, is derived from the HIV GAG protein, and is a class I and class II antigen for HIV. The flanking sequences, residues 1 to 3 of SEQ ID NO:4, CGC, and residues 19 to 22 of SEQ ID NO:4, CGCG, were added to HIV GAG sequence to provide crosslinkable moieties, that is the sulfhydryl groups linked to the α-carbon atom of the cysteines residues.

### Example 13 UV Analysis of Crosslinking Between Peptide and Block Copolymer

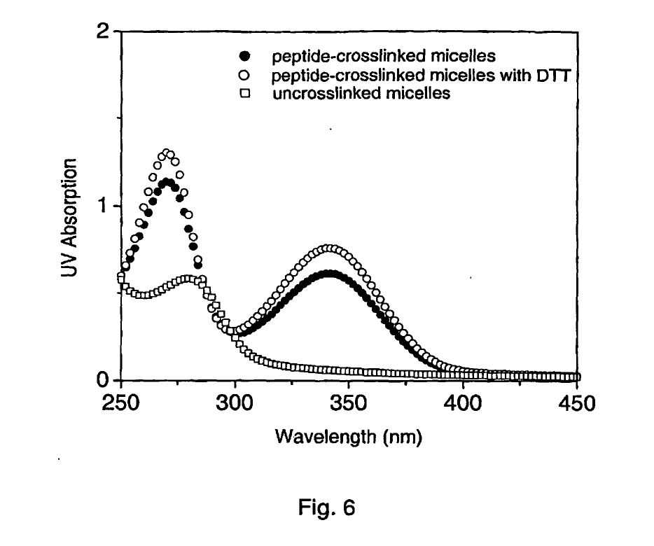

Block copolymer micelles were prepared by mixing 0.5 mg of PEG-PLL(thiopyridyl), prepared as given in Example 11, and 0.1 mg of ISS DNA with 0.5 ml of 50 mM NaH₂PO₄ buffer (PH 7.4) in a 1.5 ml microcentrifuge tube at room temperature without agitation for about 2 hours. The amounts of PEG-PLL(thiopyridyl) and ISS DNA given represent a 15:1 amine to phosphate mole ratio. After incubation for 2 hours at room temperature, 0.1 mg of the peptide described in Example 12 was added to the micelles. The amount of peptide used with the given amount of PEG-PLL(thiopyridyl) represents a 1:1 cysteine to thiopyridal ratio. The extent of the crosslinking reaction between the cysteines on the peptide with the thiopyridal groups in the PEG-PLL(thiopyridyl)/ISS DNA micelles was determined by UV analysis at 342 nm as a measure of released thiopyridone. The UV spectra of representative samples are shown in FIG. 6. The percent of cysteine groups reacted was determined by the following formula:

$$Reacted peptide ( \% ) = \frac{{ABS}_{1} - {ABS}_{2}}{{ABS}_{o}} \times 100 \%$$

wherein ABS₁ is the UV absorption at 342 nm for the reaction following peptide crosslinking of the micelles (see the filled circles in FIG. 6); ABS₂ is the UV absorption at 342 nm for the non-crosslinked micelles (see the empty squares in FIG. 6); and ABS₀ is the UV absorption at 342 nm when all of the thiopyridone groups have been released by addition of DTT, (see the empty circles in FIG. 6).

### Example 14 Rate of Crosslinking of Peptide with PEG-PLL(Thiopyridyl)/ISS DNA Micelles

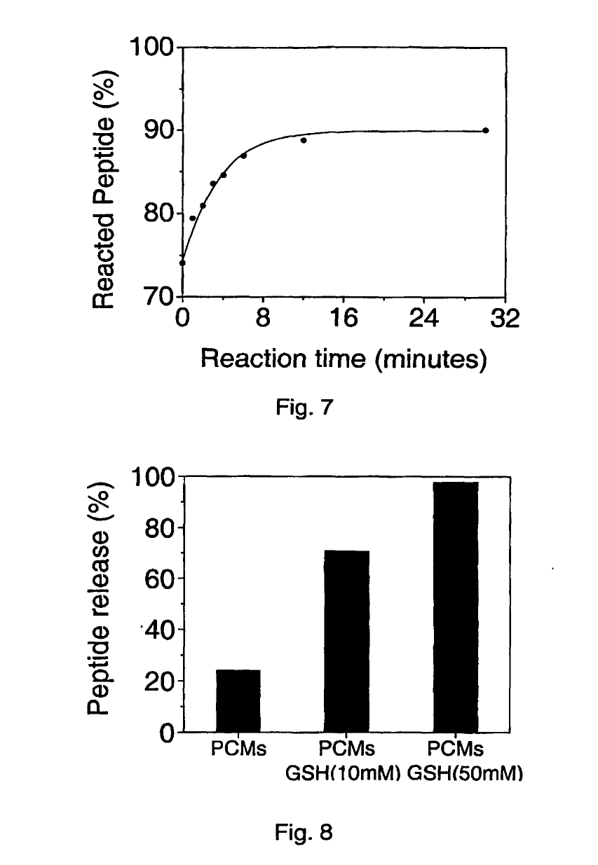

Using the UV analytical method described in Example 13, the rate of reaction of the peptide with the PEG-PLL(thiopyridyl)/ISS DNA micelles was determined. The reaction conditions are those as given in Example 13, and the reaction was terminated at various times. The results of the experiment are given in FIG. 7, and the data show that at an equimolar ratio of cysteines groups (on the peptide) to thiopyridal groups (on the PEG-PLL-thiopyridal/ISS DNA micelles) about 90% of the peptide cysteine groups have been consumed in less than an hour. The reaction reaches completion, under these conditions, in about 15-20 minutes as shown in FIG. 7.

### Example 15 Release Peptide from Peptide Crosslinked Micelles Under Reducing Conditions

PEG-PLL(thiopyridyl)/ISS DNA micelles were prepared and crosslinked with peptide as described in Example 12. The PCM prepared were then treated with various concentrations of glutathione for 24 hours at room temperature, filtered through a 20 nm filter and the amount of crosslinked peptide released determined from analysis of samples by HPLC. HPLC analysis was carried using a reverse phase column (Discovery HS C18 column, 250 mm×4.6 mm, made by Supelco) using a gradient of 15-45% acetonitrile in 0.1% aqueous trifluoroacetic acid (TFA) at 1 ml/min with a run time of 0-30 mins. Glutathione (“GST”) is a reducing agent that is capable of reducing the disulfide bonds covalently linking the peptide to the PCM. FIG. 8 shows that about 70% of the crosslinked peptide was released in the presence of 10 mM GST, and nearly 100% of the peptide released in the presence of 50 mM GST. In the absence of added reducing agent, about 20% of the peptide was released under these conditions.

### Example 16 Release of ISS DNA from Peptide Crosslinked Micelles

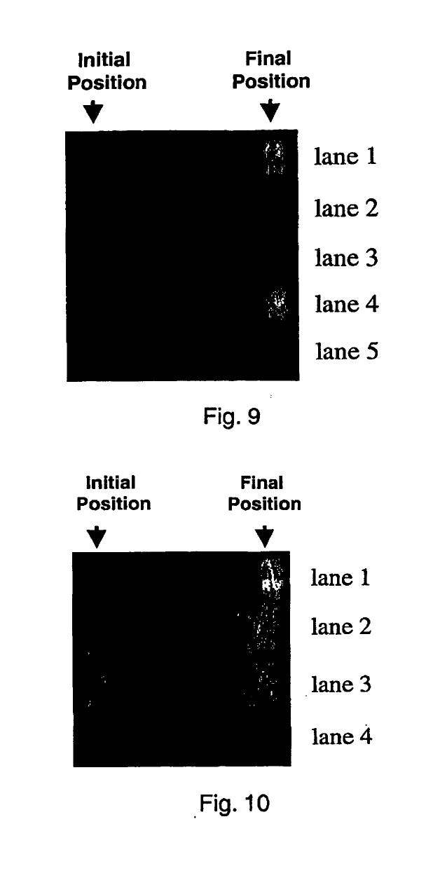

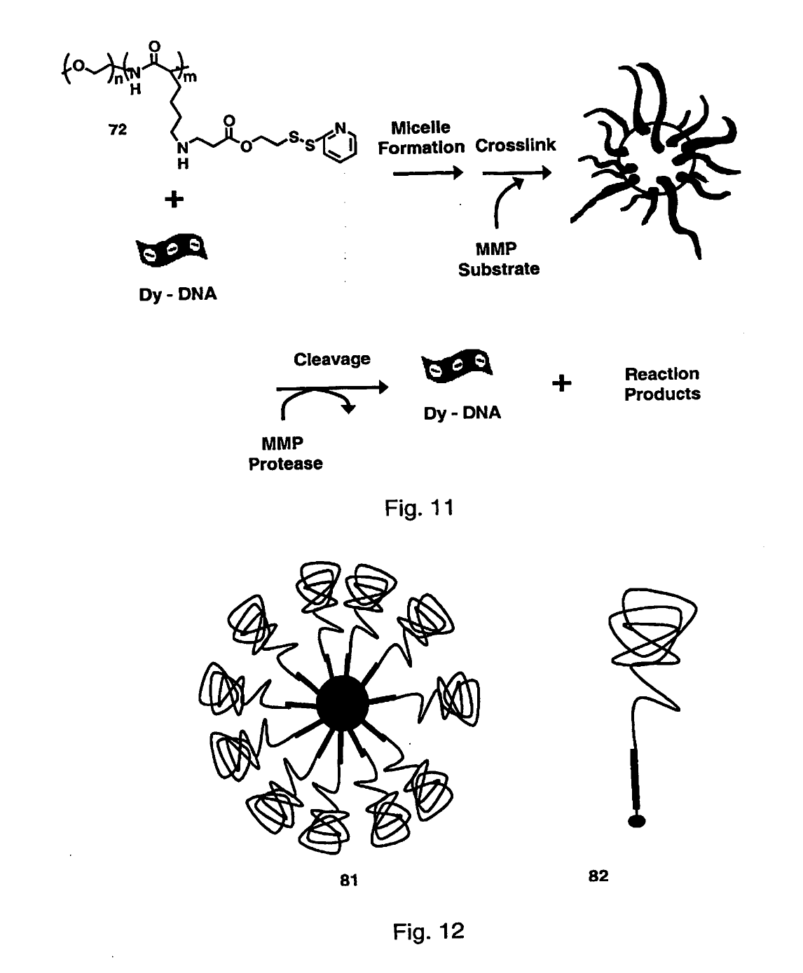

PCM were prepared as described in Example 12 and contained ISS DNA. The PCM were treated under various conditions and nature of the ISS DNA analyzed by agarose gel electrophoresis. Under the conditions of the experiment, ISS DNA retained in a micelle would not enter the gel and would not be visible in the position indicated as “final position” in FIGS. 9 and 10. However, if ISS DNA were not retained in the micelle or were released from the micelle, then it would migrate to the position indicated as “final position”. Polyvinylsulfonate (“PVS”) is a polyanion with a high charge density capable of displacing DNA from a polycation such as PLL. The data in FIG. 9 show that ISS DNA alone (see lane 1, DNA alone) migrated to the marked final position. In contrast, ISS DNA within a micelle prepared as described in Example 12 does not enter the gel and no ISS DNA is seen in the marked final position (see FIG. 9, lane 2). Similar results were obtained when the micelle was crosslinked with peptide (see FIG. 9, lane 3). In contrast, when non-crosslinked micelles were incubated in the presence of PVS prior to analysis, the ISS DNA was displaced from the micelle and migrated to the marked final position (see FIG. 9, lane 4). PVS had no effect on the displacement of the ISS DNA from micelle if the micelle was previously crosslinked with peptide (see FIG. 9, lane 5).

In order to determine if the ISS DNA could be released from the micelle if the crosslinking were reversed or degraded, peptide crosslinked micelles were incubated in the presence of GST at concentrations similar to normal intracellular concentrations of GST. FIG. 10 shows the results of this experiment as follows: Lane 1 shows that untreated, control ISS DNA migrated to the marked final position; Lane 2 shows that when PCM were treated with 10 mM GST and incubated in the presence of PVS, a significant fraction of ISS DNA was released from the PCM; Lane 3 shows conditions and results similar to those in Lane 2, except that the GST concentration was increased to 15 mM; and, Lane 4 shows that ISS DNA was not released from PCM incubated in the presence of PVS without GST.

### Example 17 Synthesis of PEG-Diacrylate

Polyethylene glycol (1.5 g, 85.9 μmol, PEG diol, HO—(CH₂CH₂O)ₙCH₂CH₂—OH, MW 35,000) was dissolved in 10 ml of CH₂Cl₂. Triethylamine (48 μl, 343.61 mmol) was added dropwise to the solution with stirring, followed by the dropwise addition of acryloyl chloride, (CAS 814-68-6, 28.0 μl, 343.6 μmol) at a temperature of about 0˜4° C. The reaction mixture was carried out for about 4 hours. The reaction solution was precipitated in cold ether for three times. The precipitant was dried under vacuum to yield 1.4 g (93.3%). The product was analyzed by ¹H NMR (D₂O) and the following data were obtained: 3.52 ppm (PEG), 5.80 ppm, 6.43 ppm (dd, 2H, CH₂═CH—COO—), 6.05 ppm(dd, 1H, CH₂═CH—COO—).

### Example 18 Synthesis of Peptide-DTPA

### Synthesis of Trypsin Substrate Peptide-DTPA

A trypsin substrate peptide with the sequence of SEQ ID NO: 5, Arg-Arg-Arg-Arg-Gly-Cys-Gly, was synthesized on an 433A Peptide Synthesizer (Applied Biosystems). The peptide was not cleaved from the synthesis resin and washed with dichloromethane prior to use in the coupling reaction. Diethylenetriaminepentaacetic acid anhydride (DTPA, CAS 67-43-6) was covalently attached to the N-terminus of the peptide by dissolving about 285 mg of DTPA in 10 ml of anhydrous dimethyl sulfoxide (DMSO). To the solution containing DTPA solution was added about 229 mg of the peptide resin. The coupling of DTPA to the peptide resing was allowed to proceed overnight at room temperature. The trypsin substrate peptide, coupled to DTPA, was then cleaved from the resin support using 1% triisopropylsilane (TIS), 2.5% ethanedithiol (EDT), 2.5% water, and 94% TFA for 2 hours at room temperature. The trypsin substrate peptide-DTPA was purified by reverse phase HPLC on a Jupiter 10μ Proteo 90A column (250 mm×10 mm, Phenomonex Corporation) using a gradient system of 4% to 50% acetonitrile in a 0.1% aqueous solution of TFA with a run time of 0 to 30 minutes. The calculated mass of 1235.4 for the trypsin substrate peptide-DTPA compound was confirmed by mass spectrometry.

### Synthesis of MMP-7 Substrate Peptide-DTPA

MMP7 substrate peptide, with the sequence of SEQ ID NO:6, Arg-Pro-Leu-Ala-Leu-Trp-Arg-Ser-Gly-Cys-Gly, was synthesized on a 433A Peptide Synthesizer (Applied Biosystems). The peptide was not removed from the solid phase synthesis resin prior to use in the following reaction. In a 25 ml round bottom flask diethylenetriaminepentaacetic dianhydride (DTPA-DA, 228 mg, 0.63 mmol) was dissolved in 10 ml DMSO, which had been previously dried over 4 molecular sieves. Solid-phase MMP7 substrate peptide (200 mg) was then added to the reaction mixture and triethylamine (87 μl, 0.63 mmol) was added drop wise with stirring. The mixture was allowed to react for 24 hours at room temperature. The reaction was washed with DMSO with gradually increasing amounts of dimethylformamide (DMF) until only pure DMF was present. The washing process was repeated with methanol, then dichloromethane. The reaction was dried under vacuum overnight. The MMP7 substrate peptide-DTPA was cleaved from the resin support using 1% TIS, 2.5% EDT, 2.5% water, and 94% TFA for 2 hours at room temperature. The peptide-DTPA was purified by reverse phase HPLC on a Jupiter 10μ Proteo 90A column (250 mm×10 mm, Phenomonex Corp) using a gradient of 20% to 50% acetonitrile in an 0.1% aqueous TFA solution with a run time of 0 to 30 mins. The calculated mass of 1592 for peptide-DTPA was confirm by mass spectrometry.

### Example 19 Synthesis of PEG-Peptide-DTPA-Dy

### Synthesis of PEG-Peptide-DTPA-Dy with Trypsin Substrate Peptide-DTPA

PEG-diacrylate (39.2 mg, 1.12 μmol) was dissolved in 200 μl of PBS buffer (200 mM, pH 7.4). Peptide-DTPA (2.0 mg, 1.62 μmol) was dissolved in 10 μl of PBS buffer (200 mM, pH 7.4), which was then added to 200 μl of PEG-diacrylate solution in PBS buffer (200 mM, pH 7.4). After 12 hours at room temperature, the reaction solution was dialyzed against water for 24 hours using a dialysis membrane with a molecular weight cut-off 3500 Da. The dialyzed solution with an approximate volume of 1 ml was then removed from the dialysis tubing and placed in a round bottom flask, and 20 mg DyCl₃ was added to this solution. The solution was stirred for 3 hours at room temperature, and then dialyzed against water for 24 hours using a dialysis membrane with a molecular weight cut-off 3500 Da. The dialyzed solution containing PEG-peptide-DTPA-Dy was freeze-dried under vacuum to isolate the PEG-peptide-DTPA-Dy.

### Synthesis of PEG-Peptide-DTPA-Dy with MMP-7 Substrate Peptide-DTPA

PEG-diacrylate (10 mg, 0.28 μmol) was dissolved in 1 ml of PBS buffer (100mM, pH 8). MMP-7 substrate Peptide-DTPA (1 mg, 0.63 μmol), prepared as described in Example 18 was added to the PEG-diacrylate solution and allowed to react for 12 hours at room temperature. After 12 hours, DyCl₃ (8 mg, 301 mmol) was added to the reaction mixture. The reaction was purified 3 hours later by dialysis (dialysis membrane cut-off MW 3500) in deionized water. Dialysis occurred overnight with two 1 L water changes. The dialyzed solution was freeze-dried to yield the final product.

PEG-peptide-DTPA-Dy micelles were prepared using the conditions described in Example 12. The formation of PEG-peptide-DTPA-Dy micelles was confirmed by dynamic light scattering measurements (Zetasizer Nano ZS, Malvern Instruments) taken on a 100 μg/ml solution of the PEG-peptide-DTPA-Dy in water which had been filtered through a 100 nm filter. These measurements determined that the PEG-peptide-DTPA-Dy forms micelles with an average diameter of 785 nm, using the Cumulant method.

R₂ of the micelles was determined as a parameter indicative of the effectiveness of these compounds at lower water T₂, and thus, their effectiveness as contrast agents. In these experiments, the higher the value of R₂, the more effective a contrast agent is at lowering T₂. The R₂ of micelles composed of PEG-peptide-DTPA-Dy was compared against PEG-DTPA-Dy, which does not form micelles. The R₂ of PEG-peptide-DTPA-Dy was 400 mM⁻¹s⁻¹, whereas the R₂ of PEG-DTPA-Dy was found to be 20 mM⁻¹s⁻¹.

### Example 20 Synthesis OF PEG-DTPA-DY

50 mg of polyethylene glycol (methoxy-PEG-amino, CH₃O—(CH₂CH₂O)ₙCH₂CH₂—NH₂, MW=20,000) and 4 mg of DTPA were dissolved in 1 ml of DMSA with 20 μl of triethyl amine. The reaction was stirred in a 5 ml round bottom flask overnight at room temperature and then dialyzed against water overnight with dialysis tubing with a molecular weight cut-off of 3,500 Da and lyophilized. The tri-nitrobenzene-sulfonic acid assay indicated that a 100% of the amines had reacted with the DTPA. 10 mg of the PEG-DTPA was dissolved in 1 ml of water and then 10 mg of DyCl₃ was added, after 3 hours of stirring at room temperature in a round bottom flask, the reaction was dialyzed overnight against deionized water using a dialysis membrane with a molecular weight cut-off of 3,500 Da and following dialysis the sample was lyophilized

### Example 21 Increase in Water T₂ Following Enzymatic Cleavage of Peptide Crosslink

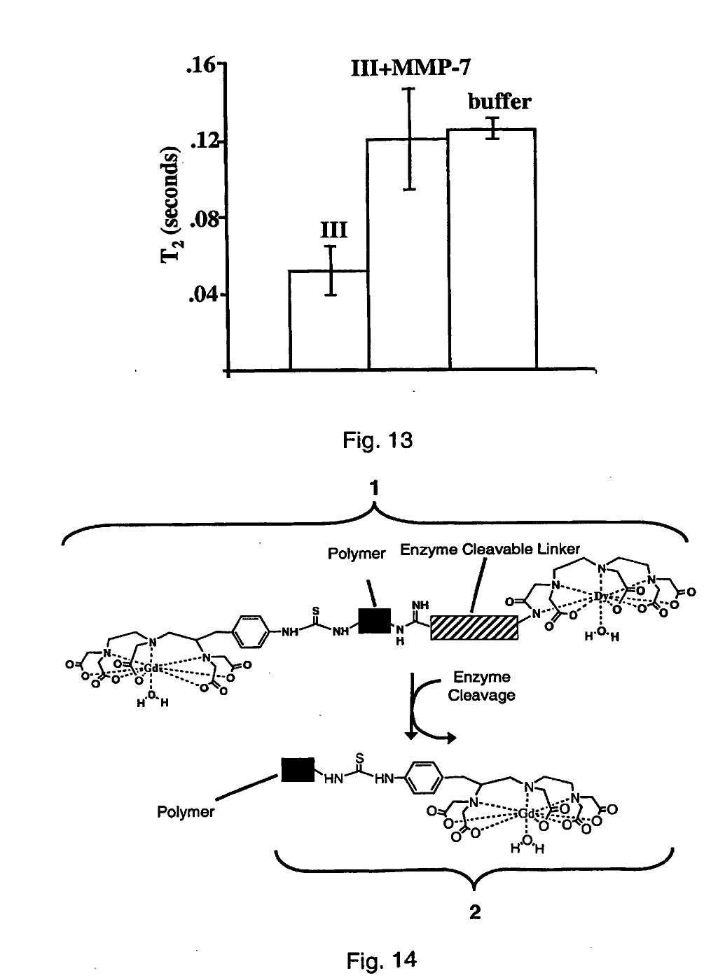

PEG-Peptide-DTPA-Dy (1.9 μM) containing MMP-7 substrated peptide-DTPA and MMP7 (0.38 μM) were added to 500 μL of buffer in D₂O (50 mM Tricine, 0.2M NaCl, 10 mM CaCl₂, 50 μM ZnSO₄, 0.005% Brij 35, pH 7.4). The reaction was performed in triplicate at 37° C. in standard NMR tubes for 4.5 hours with a total of 3 T₁/T₂ measurements each. Control solutions of PEG-Peptide-DTPA-Dy (1.9 μM), MMP7 (0.38 μM), and buffer were prepared and measured under the same conditions. Fluorescamine data was collected after 6 hours to confirm substrate cleavage. The T₂ signal was measured for MMP-7 treated micelles, untreated micelles, and for buffer without micelles. The results shown in FIG. 13 indicate that upon treatment of micelles with MMP-7 that the T₂ signal increase by over two-fold and is comparable to the signal of buffer only samples.

### Example 22 Synthesis of 2-(Pyridin-2-Yldisulfanyl)Ethanol

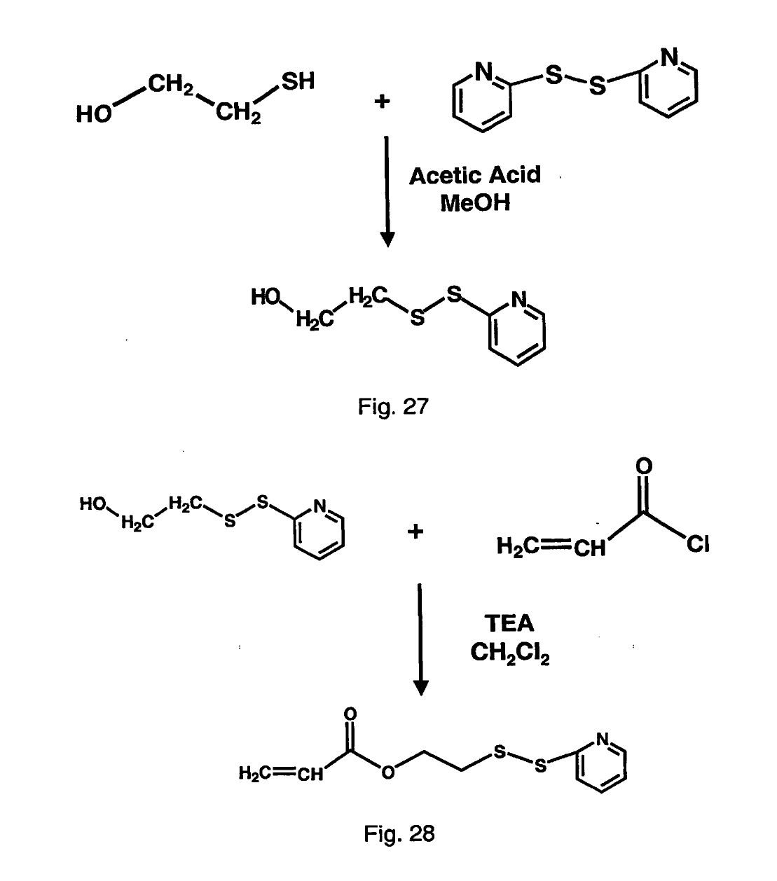

The reaction scheme for the preparation of 2-(pyridin-2-yldisulfanyl)ethanol is shown in FIG. 27. Acetic acid (1.5 ml) was added to a solution of 2,2′-dithiodipyridine (4.2 g) in methanol (15 ml). A solution of β-mercaptoethanol (0.75 ml) in methanol (25 ml) was added dropwise to the above solution. The reaction was stopped when the starting material 2,2-dithiodipyridine was consumed as detected by thin layer chromatography (TLC) The R_{f} of pyridinedithioethanol in a 1:1 mixture of hexane and ethyl acetate is 0.4. The yield of pyridinedithioethanol is 73%. The ¹H NMR spectra of pyridinedithioethanol in CDCl₃ is shown in Figure X. Molecular weight as determined by mass spectrum was 188.0. The formula for the compound is C₇H₁₀NOS₂.

### Example 23 Synthesis of 2-(Pyridin-2-Yldisulfanyl)Ethyl Acrylate

The synthetic scheme for the preparation of 2-(pyridin-2-yldisulfanyl)ethyl acrylate is shown in FIG. 28. Triethylamine (89011) was added to a solution of pyridinedithioethanol (890 μg) in CH₂Cl₂ (8 ml). Acryloyl chloride (520 μl) was added dropwise to the above solution. The reaction was stopped when the starting material pyridinedithioethanol was consumed as shown by TLC using a 1:1 solution of hexane:ethyl acetate as the mobile phase. The reaction mixture was purified by extraction with 50 ml CH₂Cl₂ and 50 ml deionized water. The extraction procedure was repeated 3 times and the organic phase was collected. After which MgSO₄ was added to the collected organic phase to remove water. The product was obtained after rotary evaporation and drying under high vacuum, with a yield of 99%. The ¹H NMR spectra of 2-(pyridin-2-yldisulfanyl)ethyl acrylate was determined and the following data obtained (CD₃Cl, 400 MHz): δ 8.47 (d, J=4.8, 1H), δ 7.67 (t, J=9.8, 1H), δ 7.61 (d, J=8.0, 1H), δ 7.09 (t, J=6.6, 1H), δ 6.41 (d, J=8.8, 1H), δ 6.10 (t, J=14.0, 1H), δ 5.81 (d, J=5.2, 1H), δ 4.41 (t, J=7.0, 2H), δ 3.08 (t, J=4.8, 2H). Molecular weight was determined using mass spectrometry as 241.9. The formula of the compound is: C₁₀H₁₁NO₂S₂.

### REFERENCES

Caravan, P., Greenfield, M. T., and Bulte, J. W. M. (2001). Molecular factors that determine curie spin relaxation in Dysprosium complexes. Magnetic Resonance in Medicine 46:917-922.
Chambers, A. F., Groom, A. C., and MacDonald, I. C. (2002). Dissemination and growth of cancer cells in metastatic sites. Nature Reviews 2:563-572.
Coussens, L. M., Fingleton, B., and Matrisian, L. M. (2002). Matrix metalloproteinase inhibitors and cancer: trials and tribulations. Science 295:2387-2393.
Giannelli, G., Bergamini, C., Marinosci, F., Fransvea, E., Quaranta, M., Lupo, L., Schiraldi, S., and Antonaci, S. (2002). Clinical Role of MMP-2/TIMP-2 Imbalance in hepatocellular carcinoma. Int. J. Cancer 97:425-431.
Gordon, M. J., Chu, K. C., Margaritis, A., Martin, A. J., Ethier, C. R., and Rutt, B. K. (1999). Measurement of Gd-DTPA diffusion through hydrogel using a novel magnetic resonance imaging method. Biotechnology and Bioengineering. 65:459-467.
Harada, A. and Kataoka, K. (1995). Formation of polyion complex micelles in an aqueous milieu from a pair of oppositely-charged block copolymers with poly(ethylene glycol) segments. Macromolecules. 28:5294-5299.
Huang, Y. W., Gupta, V. K. (2002). Influence of polymer flux and chain length on adsorption of poly(ethylene oxide) on physically heterogeneous surfaces. Langmuir. 18:2280-2287.
Pila\check{r}, J., and Labský, J. (1991a). EPR study of chain rotational dynamics in dilute aqueous solutions of spin-labeld poly(methacrylic acid) at different degrees of neutralization. Macromolecules. 24:4188-4194.
Pila\check{r}, J., and Labský, J. (1991b). EPR study of chain rotational dynamics in dilute aqueous solutions of spin-labeld poly(methacrylic acid) at different degrees of neutralization. Macromolecules. 27:3977-3981.
Pluen, A., Netti, P. A., Jain, R. K., and Berk, D. A. (1999). Diffusion of macromolecules in agarose gels: comparison of linear and globular configurations. Biophysical Journal 77:542-552.
Ramanujan, S., Pluen, A., McKee, T. D., Brown, E. B., Boucher, Y., and Jain, R. K. (2002). Diffusion and convection in collagen gels: implications for transport in the tumor interstitium. Biophysical Journal 83:1650-1660.
Ramos-DeSimone, N., Hahn-Dantona, E., Sipley, J., Nagase, H., French, D. L., and Quigley, J. P. (1999). Activation of matrix metalloproteinase-9 (MMP-9) via a converging plasmin/stromelysin-1 cascade enhances tumor cell invasion. Journal of Biological Chemistry 274:13066-13076.
Tutton, M. G., George, M. L., Eccles, S. A., Burton, S., Swift, R. I., and Abulafi, A. M. (2003). Use of plasma MMP-2 and MMP-9 levels as a surrogate for tumor expression in colorectal cancer patients. In. J. Cancer 107:541-550.
Vander Elst, L., Zhang, S., Sherry, A. D., Laurent, S. S., Botterman, F., and Muller, R. N. (2002). Dy-complexes as high field T₂ contrast agents: influence of water exchange rates. Acad. Radiol. 9 (Suppl 2), S297-S299.

1. A detectable construct, comprising, at least one detectable agent covalently bonded to at least one of a linking region or a reaction region, wherein the linking region is a polymer and the reaction region is altered by bioactivity.

2. The detectable construct of claim 1, wherein the detectable agent comprises an organic molecule, metal ion, salt or chelate, cluster, particle, inorganic dye, luminescent meal complex, or radiolabeled peptide, protein or polymer.

3. The detectable construct of claim 2, wherein the detectable agent comprises a chelator molecule and a metal ion.

4. The detectable construct of claim 3, wherein the chelator molecule is DTPA, DOTA, DTPA, HP-DO3A, DTPA-BMA, enterobactin, MECAMS, EDTA or chemically modified forms of DTPA, DOTA, DTPA, DTPA-BMA, enterobactin, MECAMS, EDTA or HP-DO3A.

5. The detectable construct of claim 2, wherein the metal ion is a paramagnetic ion.

6. The detectable construct of claim 4, wherein the paramagnetic ion is Gd(III), Fe(III), Mn(II), Mn(III), Cr(III), Cu(II), Dy(III), Y(III), Tb(III), Ho(III), Er(III) or Eu(III).

7. (canceled)

8. (canceled)

9. The detectable construct of claim 1, wherein the detectable construct comprises two detectable agents, and the detectable agents are the same detectable agents.

10. The detectable construct of claim 1, wherein the detectable construct comprises two detectable agents, and the detectable agents are different detectable agents.

11. (canceled)

12. (canceled)

13. (canceled)

14. (canceled)

15. (canceled)

16. (canceled)

17. The detectable construct of claim 1, wherein the polymer is PEG.

18. The detectable construct of claim 1, wherein the polymer is poly(methacrylic acid).

19. (canceled)

20. The detectable construct of claim 1, wherein the reaction region is an enzyme substrate, a chemically reactive combination of elements, one of a pair of binding partners, a nucleic acid polymer, or a photocleaveable moiety.

21. (canceled)

22. (canceled)

23. (canceled)

24. The detectable construct of claim 1, wherein the detectable construct comprises a detectable agent covalently bonded to a linking region and the linking region is covalently bonded to a reaction region.

25. The detectable construct of claim 1, wherein the detectable construct comprises a first detectable agent covalently bonded to a linking region and the linking region is covalently bonded to a reaction region and the reaction region is covalently bonded to a second detectable agent.

26. The detectable construct of claim 24, wherein the first or second detectable agent comprises a chelator molecule and a paramagnetic metal ion.

27. (canceled)

28. The detectable construct of claim 26, wherein the detectable agent comprises the metal ion Gd(III) and the chelator molecule is DTPA.

29. The detectable construct of claim 26, wherein the detectable agent comprises the metal ion Dy(III) and the chelator molecule is DTPA.

30. The detectable construct of claim 1, wherein the detectable construct comprises a molecule, a hydrogel or a micelle.

31. (canceled)

32. (canceled)

33. (canceled)

34. (canceled)

35. (canceled)

36. (canceled)

37. (canceled)

38. (canceled)

39. (canceled)

40. (canceled)

41. The detectable construct of claim 1, further comprising a pharmaceutically acceptable formulation.

42. (canceled)

43. (canceled)

44. (canceled)

45. (canceled)

46. (canceled)

47. (canceled)

48. (canceled)

49. A method for diagnosing a bioactivity in living cells, comprising,
administering an effective amount of a detectable composition to living cells wherein the detectable composition comprises at least one detectable construct comprising at least one detectable agent covalently bonded to at least one of a linking region or a reaction region, wherein the linking region is a polymer and the reaction region is altered by bioactivity;
altering the reaction region of the detectable construct by the bioactivity,
measuring the alteration in the reaction region.

50. The method of claim 49, wherein the measuring of the alteration is by imaging methods.

51. The method of claim 49, wherein the imaging methods is magnetic resonance imaging methods.

52. A micelle comprising amphiphilic molecules comprising pendant side chains cross-linked to a reactive region.

53. The micelle of claim 52, wherein the reaction region is an antigen, active agent, detectable agent, an enzyme substrate, a chemically reactive combination of elements, one of a pair of binding partners, a nucleic acid polymer, or a photocleaveable moiety.

54. The micelle of claim 52, further comprising an active agent in the core of the micelle.

55. The micelle of claim 52, wherein the amphiphilic molecules are block copolymers of (A)n-block-(B)m, where A is a hydrophobic polymer and B is a hydrophilic polymer, the reaction region is an enzyme substrate, and the active agent is a detectable agent.
